(19) United States
(12) Patent Application Publication
(10) Pub. No.: US 2010/0203517 A1
(43) Pub. Date: Aug. 12, 2010

(54) HUMAN DIABETES SUSCEPTIBILITY PEBP4 GENE

(75) Inventors: Anne Philippi, St. Fargeau Ponthierry (FR); Jorg Hager, Mennecy (FR); Francis Rousseau, Savigny Sur Orge (FR)

Correspondence Address: OCCHIUTI ROHLICEK & TSAO, LLP, 10 FAWCETT STREET, CAMBRIDGE, MA, 02138, US

(73) Assignee: Integragen, Evry (FR)

(21) Appl. No.: 12/526,287

(22) PCT Filed: Feb. 20, 2008
(86) PCT No.: PCT/EP08/52088

§ 371 Date: Mar. 9, 2010

Related U.S. Application Data
(60) Provisional application No. 60/902,578, filed on Feb. 21, 2007.

(51) Int. Cl. C12Q 1/68

(52) U.S. Cl. 435/6

(57) ABSTRACT

The present invention relates to a diagnostic method of determining whether a subject, preferably an obese subject, is at risk of developing type 2 diabetes, which method comprises detecting the presence of an alteration in the PEBP4 gene locus in a biological sample of said subject.

The present invention relates to a method for determining a predisposition to diabetes in patients.

### BACKGROUND OF THE INVENTION

According to the new etiologic classification of diabetes mellitus, four categories are differentiated: type 1 diabetes, type 2 diabetes, other specific types, and gestational diabetes mellitus (ADA, 2003). In the United States, Canada, and Europe, over 80% of cases of Diabetes are due to type 2 diabetes, 5 to 10% to type 1 diabetes, and the remainder to other specific causes.

In Type 1 diabetes, formerly known as insulin-dependent, the pancreas fails to produce the insulin which is essential for survival. This form develops most frequently in children and adolescents, but is being increasingly diagnosed later in life. Type 2 diabetes mellitus, formerly known as non-insulin dependent diabetes mellitus (NIDDM), or adult onset Diabetes, is the most common form of diabetes, accounting for approximately 90-95% of all diabetes cases. Type 2 diabetes is characterized by insulin resistance of peripheral tissues, especially muscle and liver, and primary or secondary insufficiency of insulin secretion from pancreatic beta-cells. Type 2 diabetes is defined by abnormally increased blood glucose levels and diagnosed if the fasting blood glucose level is superior to 126 mg/dl (7.0 mmol/l) or blood glucose levels are superior to 200 mg/dl (11.0 mmol/l) 2 hours after an oral glucose uptake of 75 g (oral glucose tolerance test, OGTT). Pre-diabetic states with already abnormal glucose values are defined as fasting hyperglycemia (FH)>6.1 mmol/l and <7.0 mmol/l or impaired glucose tolerance (IGT)>7.75 mmol/l and <11.0 mmol/12 hours after an OGTT.

TABLE 1
Classification of Type 2 diabetes (WHO, 2006)

| | Fasting blood glucose | | 2 hours after an |
|---|---|---|---|
| Classification | level (mmol/l) | | OGTT (mmol/l) |
| Normo glycemia | <7.0 | and | <11.0 |
| FH only | >6.1 to <7.0 | and | <7.75 |
| IGT only | <6.1 | and | ≧7.75 to <11.0 |
| FH and IGT | >6.1 to <7.0 | and | ≧7.75 to <11.0 |
| Type 2 diabetes | ≧7.0 | or | ≧11.0 |

In 2000, there were approximately 171 million people, worldwide, with type 2 diabetes. The number of people with type 2 diabetes will expectedly more than double over the next 25 years, to reach a total of 366 million by 2030 (WHO/IDF, 2006). Most of this increase will occur as a result of a 150% rise in developing countries. In the US 7% of the general population are considered diabetic (over 15 million diabetics and an estimated 15 million people with impaired glucose tolerance).

Twin and adoption studies, marked ethnic differences in the incidence and prevalence of type 2 diabetes and the increase in incidence of type 2 diabetes in families suggest that heritable risk factors play a major role in the development of the disease. Known monogenic forms of diabetes are classified in two categories: genetic defects of the beta cell and genetic defects in insulin action (ADA, 2003). The diabetes forms associated with monogenetic defects in beta cell function are frequently characterized by onset of hyperglycemia at an early age (generally before age 25 years). They are referred to as maturity-onset diabetes of the Young (MODY) and are characterized by impaired insulin secretion with minimal or no defects in insulin action (Herman W H et al, 1994; Clement K et all, 1996; Byrne M M et all, 1996). They are inherited in an autosomal dominant pattern. Abnormalities at three genetic loci on different chromosomes have been identified to date. The most common form is associated with mutation on chromosome 12q in the locus of hepatic transcription factor referred to as hepatocyte nuclear factor (HNF)-1α (Vaxillaire M et all, 1995; Yamagata et all, 1996). A second form is associated with mutations in the locus of the glucokinase gene on chromosome 7q and result in a defective glucokinase molecule (Froguel P et all, 1992; Vionnet N et all, 1992). Glucokinase converts glucose to glucose-6-phosphase, the metabolism of which, in turn, stimulates insulin secretion by the beta cell. Because of defects in the glucokinase gene, increased plasma levels of glucose are necessary to elicit normal levels of insulin secretion. A third form is associated with a mutation in the HnfMa gene on chromosome 20q (Bell G I et all, 1991; Yamagata K et all, 1996). HNF-4α is a transcription factor involved in the regulation of the expression of HNF-4α. Point mutations in mitochondrial DNA can cause diabetes mellitus primarily by impairing pancreatic beta cell function (Reardon W et all, 1992; VanDen Ouwenland J M W et all, 1992; Kadowaki T et all, 1994). There are unusual causes of diabetes that result from genetically determined abnormalities of insulin action. The metabolic abnormalities associated with mutation of the insulin receptor may range from hyperinsulinemia and modest hyperglycemia to severe diabetes (Kahn C R et all, 1976; Taylor S I, 1992). Type 2 diabetes is a major risk factor for serious micro- and macro-vascular complications. The two major diabetic complications are cardiovascular disease, culminating in myocardial infarction. 50% of diabetics die of cardiovascular disease (primarily heart disease and stroke) and diabetic nephropathy. Diabetes is among the leading causes of kidney failure. 10-20% of people with diabetes die of kidney failure. Diabetic retinopathy is an important cause of blindness, and occurs as a result of long-term accumulated damage to the small blood vessels in the retina. After 15 years of diabetes, approximately 2% of people become blind, and about 10% develop severe visual impairment. Diabetic neuropathy is damage to the nerves as a result of diabetes, and affects up to 50% of all diabetics. Although many different problems can occur as a result of diabetic neuropathy, common symptoms are tingling, pain, numbness, or weakness in the feet and hands. Combined with reduced blood flow, neuropathy in the feet increases the risk of foot ulcers and eventual limb amputation.

The two main contributors to the worldwide increase in prevalence of diabetes are population ageing and urbanization, especially in developing countries, with the consequent increase in the prevalence of obesity (WHO/IDF, 2006). Obesity is associated with insulin resistance and therefore a major risk factor for the development of type 2 diabetes. Obesity is defined as a condition of abnormal or excessive accumulation of adipose tissue, to the extent that health may be impaired. The body mass index (BMI; kg/m²) provides the most useful, albeit crude, population-level measure of obesity. Obesity has also been defined using the WHO classification of the different weight classes for adults.

TABLE 2
Classification of overweight in adults
according to BMI (WHO, 2006)

| Classification | BMI (kg/m²) | Risk of co-morbidities |
|---|---|---|
| Underweight | <18.5 | Low (but risks of other |
| | | clinical problems increased) |
| Normal range | 18.5-24.9 | Average |
| Overweight | ≧25 | |
| Pre-obese | 25-29.9 | Increased |
| Obese class I | 30-34.9 | Moderate |
| Obese class II | 35-39.9 | Severe |
| Obese class III | ≧40 | Very severe |

More than 1 billion adults world-wide are considered overweight, with at least 300 million of them being clinically obese. Current obesity levels range from below 5% in China, Japan and certain African nations, to over 75% in urban Samoa. The prevalence of obesity is 10-25% in Western Europe and 20-27% in the Americas (WHO, 2006).

The rigorous control of balanced blood glucose levels is the foremost goal of all treatment in type 2 diabetes be it preventative or acute. Clinical intervention studies have shown that early intervention to decrease both obesity and/or pre-diabetic glucose levels through medication or lifestyle intervention, can reduce the risk to develop overt type 2 diabetes by up to 50% (Knowler W C et al, 2002). However, only 30% of obese individuals develop type 2 diabetes and the incentive for radical lifestyle intervention is often low as additional risk factors are lacking. Also, the diagnosis of type 2 diabetes through fasting blood glucose is insufficient to identify all individuals at risk for type 2 diabetes.

A further obstacle to rapidly achieve a balanced glucose homeostasis in diabetic patients is the multitude of therapeutic molecules with a wide range of response rates in the patients. Type 2 diabetes is treated either by oral application of anti-glycemic molecules or insulin injection. The oral antidiabetics either increase insulin secretion from the pancreatic beta-cells or that reduce the effects of the peripheral insulin resistance. Multiple rounds of differing treatments before an efficient treatment is found significantly decreases the compliance rates in diabetic patients.

Molecular and especially genetic tests hold the potential of identifying at risk individuals early, before onset of clinical symptoms and thereby the possibility for early intervention and prevention of the disease. They may also be useful in guiding treatment options thereby short-circuiting the need for long phases of sub-optimal treatment. Proof-of-principle has been shown for the treatment of individuals with maturity-onset diabetes of the young (MODY). Following molecular diagnosis many individuals with MODY3 or MODY2 can be put off insulin therapy and instead be treated with sulfonylureas (MODY 3) or adapted diet (MODY 2) respectively. Therefore, there is a need for a diagnostic test capable of evaluating the genetic risk factor associated with this disease. Such a test would be of great interest in order to adapt the lifestyle of people at risk and to prevent the onset of the disease.

### SUMMARY OF THE INVENTION

The present invention now discloses the identification of a diabetes susceptibility gene.

The invention thus provides a diagnostic method of determining whether a subject is at risk of developing type 2 diabetes, which method comprises detecting the presence of an alteration in the PEBP4 gene locus in a biological sample of said subject.

Specifically the invention pertains to single nucleotide polymorphisms in the PEBP4 gene on chromosome 6 associated with type 2 diabetes and body weight.

### LEGEND TO THE FIGURES

FIG. 1: High density mapping using Genomic Hybrid Identity Profiling (GenomeHIP). Graphical presentation of the linkage peak on chromosome 8p22-p21.2. The curve depicts the linkage results for the GenomeHip procedure in the region. A total of 7 Bac clones on human chromosome 8 ranging from position p-ter-17.513.477 to 26.476.264-cen were tested for linkage using GenomeHip. Each point on the x-axis corresponds to a clone. Significant evidence for linkage was calculated for clone BACAl2ZC07 (p-value 1.9E-10). The whole linkage region encompasses a region from 19.417.224 base pairs to 25.245.630 base pairs on human chromosome 8. The p-value less to 2×10⁻⁵ corresponding to the significance level for significant linkage was used as a significance level for whole genome screens as proposed by Lander and Kruglyak (1995).

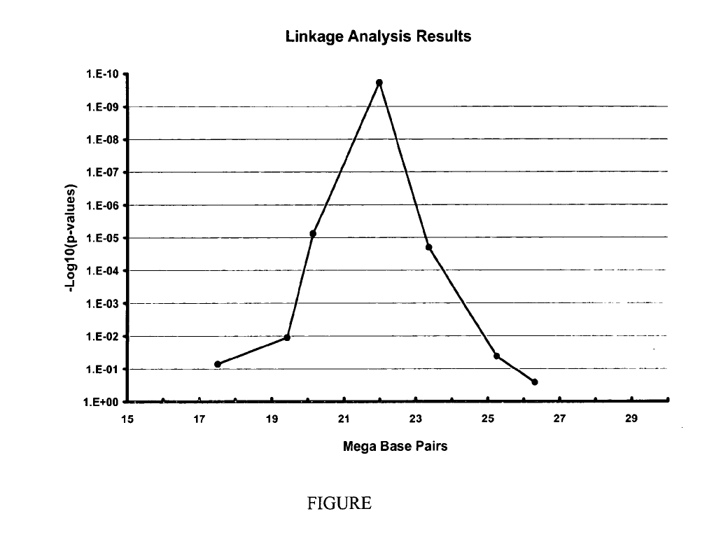

### DETAILED DESCRIPTION OF THE INVENTION

The present invention discloses the identification of PEBP4 as a diabetes susceptibility gene in individuals with type 2 diabetes. More specifically the invention pertains to individuals with both type 2 diabetes and a BMI>27 kg/m². Various nucleic acid samples from diabetes families were submitted to a particular GenomeHiP process. This process led to the identification of particular identical-by-descent (IBD) fragments in said populations that are altered in diabetic subjects with a BMI>27 kg/m². By screening of the IBD fragments, the inventors identified the PEBP4 gene as a candidate for type 2 diabetes. SNPs of the PEBP4 gene were also identified, as being associated to type 2 diabetes, more particularly in obese subjects.

### DEFINITIONS

Type 2 diabetes is characterized by chronic hyperglycemia caused by pancreatic insulin secretion deficiency and/or insulin resistance of peripheral insulin sensitive tissues (e.g. muscle, liver). Long term hyperglycemia has been shown to lead to serious damage to various tissue including nerves tissue and blood vessels. Type 2 diabetes accounts for 90% all diabetes mellitus cases around the world (10% being type 1 diabetes characterized by the auto-immune destruction of the insulin producing pancreatic beta-cells). The invention described here pertains to a genetic risk factor for individuals to develop type 2 diabetes. Preferably the invention describes increased risk for overweight individuals (BMI>27 kg/m²).

Within the context of this invention, the PEBP4 gene locus designates all PEBP4 sequences or products in a cell or organism, including PEBP4 coding sequences, PEBP4 non-coding sequences (e.g., introns), PEBP4 regulatory sequences controlling transcription and/or translation (e.g., promoter, enhancer, terminator, etc.), as well as all corresponding expression products, such as PEBP4 RNAs (e.g., mRNAs) and PEBP4 polypeptides (e.g., a pre-protein and a mature protein). The PEBP4 gene locus also comprise surrounding sequences of the PEBP4 gene which include SNPs that are in linkage disequilibrium with SNPs located in the PEBP4 gene.

As used in the present application, the term “PEBP4 gene” designates the gene phosphatidylethanolamine-binding protein 4, as well as variants or fragments thereof, including alleles thereof (e.g., germline mutations) which are related to susceptibility to type 2 diabetes. The PEBP4 gene may also be referred to as CORK-1, CORK1, GWTM1933, MGC22776, PRO44081. It is located on chromosome 8 at position 8p21.3. The cDNA sequence is shown as SEQ ID NO:1, and the protein as SEQ ID NO:2 (EMBL NM144962).

PEBP4 is expressed in most human tissues and highly expressed in tumor cells. Its expression in tumor cell is further enhanced upon tumor necrosis factor (TNF) a treatment, whereas PEBP4 normally co-localizes with lysosomes, TNFα stimulation triggers its transfer to the cell membrane, where it binds to Raf-1 and Mek1. L929 cells over-expressing PEBP4 are resistant to both TNFα-induced apoptosis. Co-precipitation and in vitro protein binding assay demonstrated that PEBP4 interacts with Raf-1 and MERK1. A truncated from of PEBP4, lacking the PE-binding domain, maintains lysosomal co-localization but has no effect on cellular responses to TNFα. Given that MCF-7 breast cancer cells expressed PEBP4 at a high level, small interfering RNA was used to silence the expression of PEBP4. We demonstrated that down-regulation of PEBP4 expression sensitizes MCF-7 breast cancer cells to TNFα-induced apoptosis. PEBP4 appears to promote cellular resistance to TNF-induced apoptosis by inhibiting activation of the Raf-1/MEK/ERK pathway, JNK, and PE externalization, and the conserved region of PE-binding domain appears to play a vital role in this biological activity of PEBP4 (Wang X. et al, 2004).

The term “gene” shall be construed to include any type of coding nucleic acid, including genomic DNA (gDNA), complementary DNA (cDNA), synthetic or semi-synthetic DNA, as well as any form of corresponding RNA.

The PEBP4 variants include, for instance, naturally-occurring variants due to allelic variations between individuals (e.g., polymorphisms), mutated alleles related to diabetes, alternative splicing forms, etc. The term variant also includes PEBP4 gene sequences from other sources or organisms. Variants are preferably substantially homologous to SEQ ID No 1, i.e., exhibit a nucleotide sequence identity of at least about 65%, typically at least about 75%, preferably at least about 85%, more preferably at least about 95% with SEQ ID No 1. Variants of a PEBP4 gene also include nucleic acid sequences, which hybridize to a sequence as defined above (or a complementary strand thereof) under stringent hybridization conditions. Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

A fragment of a PEBP4 gene designates any portion of at least about 8 consecutive nucleotides of a sequence as disclosed above, preferably at least about 15, more preferably at least about 20 nucleotides, further preferably of at least 30 nucleotides. Fragments include all possible nucleotide lengths between 8 and 100 nucleotides, preferably between 15 and 100, more preferably between 20 and 100.

A PEBP4 polypeptide designates any protein or polypeptide encoded by a PEBP4 gene as disclosed above. The term “polypeptide” refers to any molecule comprising a stretch of amino acids. This term includes molecules of various lengths, such as peptides and proteins. The polypeptide may be modified, such as by glycosylations and/or acetylations and/or chemical reaction or coupling, and may contain one or several non-natural or synthetic amino acids. A specific example of a PEBP4 polypeptide comprises all or part of SEQ ID No: 2.

### Diagnosis

The invention now provides diagnosis methods based on a monitoring of the PEBP4 gene locus in a subject. Within the context of the present invention, the term ‘diagnosis” includes the detection, monitoring, dosing, comparison, etc., at various stages, including early, pre-symptomatic stages, and late stages, in adults or children. Diagnosis typically includes the prognosis, the assessment of a predisposition or risk of development, the characterization of a subject to define most appropriate treatment (pharmacogenetics), etc.

The present invention provides diagnostic methods to determine whether a subject, more particularly an obese subject, is at risk of developing type 2 diabetes resulting from a mutation or a polymorphism in the PEBP4 gene locus.

It is therefore provided a method of detecting the presence of or predisposition to type 2 diabetes in a subject, the method comprising detecting in a biological sample from the subject the presence of an alteration in the PEBP4 gene locus in said sample. The presence of said alteration is indicative of the presence or predisposition to type 2 diabetes. Optionally, said method comprises a preliminary step of providing a sample from a subject. Preferably, the presence of an alteration in the PEBP4 gene locus in said sample is detected through the genotyping of a sample.

In a preferred embodiment, said alteration is one or several SNP(s) or a haplotype of SNPs associated with type 2 diabetes. More preferably, said SNP associated with type 2 diabetes is as shown in Table 3A, i.e. said SNP is selected from the group consisting of SNP224, SNP225, and SNP244.

Other SNP(s), as listed in Table 3B, may be informative too.

TABLE 3A
SNPs on PEBP4 gene associated with type 2 diabetes:

| | | | | | | Nucleotide | | |
|---|---|---|---|---|---|---|---|---|
| | | | | | | position in | | |
| | | | | | | genomic | | |
| | | | | Frequence | Frequence | sequence of | | |
| | | | | Allele1 | Allele2 | chromosome 8 | | |
| SNP | dbSNP | | | from | From | based on NCBI | Position in | |
| identity | reference | Allele1 | Allele2 | CEU HapMap | CEU HapMap | Build 35 | locus | SEQ ID No |
| 181 | rs11781835 | A = 1 | G = 2 | 0.383 | 0.617 | 22.663.974 | Intron3 | 3 |
| 184 | rs7846693 | C = 1 | T = 2 | 0.816 | 0.184 | 22.673.257 | Intron3 | 4 |
| 193 | rs17088624 | A = 1 | G = 2 | 0.474 | 0.526 | 22.703.246 | Intron3 | 5 |
| 194 | rs10102337 | A = 1 | G = 2 | 0.4 | 0.6 | 22.704.701 | Intron3 | 6 |
| 204 | rs10503720 | C = 1 | T = 2 | 0.822 | 0.178 | 22.729.661 | Intron3 | 7 |
| 208 | rs2063688 | A = 1 | G = 2 | 0.175 | 0.825 | 22.754.035 | Intron2 | 8 |
| 211 | rs11135681 | C = 1 | T = 2 | 0.367 | 0.633 | 22.759.278 | Intron2 | 9 |
| 214 | rs11779201 | A = 1 | G = 2 | 0.182 | 0.818 | 22.763.374 | Intron2 | 10 |
| 217 | rs4284046 | A = 1 | G = 2 | 0.142 | 0.858 | 22.768.022 | Intron2 | 11 |
| 219 | rs11777000 | A = 1 | C = 2 | 0.367 | 0.633 | 22.772.871 | Intron2 | 12 |
| 222 | rs2457436 | A = 1 | G = 2 | 0.267 | 0.733 | 22.786.027 | Intron2 | 13 |
| 223 | rs11785590 | C = 1 | T = 2 | 0.15 | 0.85 | 22.792.914 | Intron2 | 14 |
| 224 | rs937969 | C = 1 | T = 2 | 0.625 | 0.375 | 22.794.051 | Intron2 | 15 |
| 225 | rs11784354 | C = 1 | T = 2 | 0.725 | 0.275 | 22.796.270 | Intron2 | 16 |
| 228 | rs2466180 | C = 1 | T = 2 | 0.858 | 0.142 | 22.802.884 | Intron2 | 17 |
| 230 | rs11135684 | A = 1 | G = 2 | 0.158 | 0.842 | 22.812.071 | Intron2 | 18 |
| 232 | rs17088737 | C = 1 | T = 2 | 0.161 | 0.839 | 22.817.408 | Intron2 | 19 |
| 234 | rs13249266 | C = 1 | T = 2 | 0.342 | 0.658 | 22.819.727 | Intron2 | 20 |
| 235 | rs2466208 | C = 1 | T = 2 | 0.408 | 0.592 | 22.822.494 | Intron2 | 21 |
| 238 | rs7010513 | C = 1 | G = 2 | 0.242 | 0.758 | 22.825.320 | Intron2 | 22 |
| 241 | rs17757261 | A = 1 | G = 2 | 0.758 | 0.242 | 22.828.329 | Intron2 | 23 |
| 244 | rs2466213 | A = 1 | C = 2 | 0.558 | 0.442 | 22.830.613 | Intron2 | 24 |
| 245 | rs11775299 | C = 1 | G = 2 | 0.7 | 0.3 | 22.830.819 | Intron2 | 25 |
| 253 | rs17088800 | A = 1 | G = 2 | 0.289 | 0.711 | 22.838.728 | Intron2 | 26 |
| 255 | rs2457426 | A = 1 | C = 2 | 0.608 | 0.392 | 22.842.095 | 5′ | 27 |

TABLE 3B
Additional SNPs on PEBP4 gene:

| | | | | | | Nucleotide | | |
|---|---|---|---|---|---|---|---|---|
| | | | | | | position in | | |
| | | | | | | genomic | | |
| | | | | Frequence | Frequence | sequence of | | |
| | | | | Allele1 | Allele2 | chromosome 8 | | |
| SNP | dbSNP | | | from | From | based on NCBI | Position in | |
| identity | reference | Allele1 | Allele2 | CEU HapMap | CEU HapMap | Build 35 | locus | SEQ ID No |
| 159 | rs4872536 | G = 1 | T = 2 | 0.237 | 0.763 | 22.625.048 | 3′ | 28 |
| 160 | rs1047398 | C = 1 | T = 2 | 0.417 | 0.583 | 22.626.963 | Intron 6 | 29 |
| 161 | rs2280107 | A = 1 | G = 2 | 0.792 | 0.208 | 22.627.411 | Intron 6 | 30 |
| 162 | rs6558179 | C = 1 | G = 2 | 0.683 | 0.317 | 22.628.695 | Intron 6 | 31 |
| 163 | rs4872539 | C = 1 | T = 2 | 0.383 | 0.617 | 22.632.507 | Intron 6 | 32 |
| 164 | rs17088571 | A = 1 | G = 2 | 0.108 | 0.892 | 22.633.948 | Intron 6 | 33 |
| 166 | rs1877673 | C = 1 | T = 2 | 0.817 | 0.183 | 22.638.219 | Intron6 | 34 |
| 167 | rs1129474 | A = 1 | G = 2 | 0.542 | 0.458 | 22.640.663 | Intron3 | 35 |
| 168 | rs7812900 | C = 1 | T = 2 | 0.708 | 0.292 | 22.643.532 | Intron3 | 36 |
| 169 | rs7001279 | A = 1 | C = 2 | 0.233 | 0.767 | 22.646.724 | Intron3 | 37 |
| 170 | rs6558183 | A = 1 | T = 2 | 0.775 | 0.225 | 22.648.953 | Intron3 | 38 |
| 171 | rs13255912 | A = 1 | G = 2 | 0.175 | 0.825 | 22.651.773 | Intron3 | 39 |
| 172 | rs7841894 | C = 1 | T = 2 | 0.758 | 0.242 | 22.652.463 | Intron3 | 40 |
| 173 | rs7013424 | A = 1 | G = 2 | 0.708 | 0.292 | 22.653.259 | Intron3 | 41 |
| 174 | rs7845221 | C = 1 | T = 2 | 0.683 | 0.317 | 22.655.366 | Intron3 | 42 |
| 175 | rs2048651 | A = 1 | G = 2 | 0.343 | 0.657 | 22.656.898 | Intron3 | 43 |
| 176 | rs4467934 | A = 1 | T = 2 | 0.692 | 0.308 | 22.657.076 | Intron3 | 44 |
| 177 | rs10780147 | G = 1 | T = 2 | 0.692 | 0.308 | 22.657.543 | Intron3 | 45 |
| 178 | rs11781095 | A = 1 | G = 2 | 0.831 | 0.169 | 22.657.732 | Intron3 | 46 |
| 179 | rs4872541 | A = 1 | C = 2 | 0.6 | 0.4 | 22.657.991 | Intron3 | 47 |
| 180 | rs1877674 | A = 1 | G = 2 | 0.407 | 0.593 | 22.662.237 | Intron3 | 48 |
| 182 | rs12682145 | A = 1 | G = 2 | 0.792 | 0.208 | 22.669.117 | Intron3 | 49 |
| 183 | rs1028056 | G = 1 | T = 2 | 0.742 | 0.258 | 22.670.908 | Intron3 | 50 |
| 185 | rs10503719 | C = 1 | T = 2 | 0.775 | 0.225 | 22.673.848 | Intron3 | 51 |
| 186 | rs11984824 | C = 1 | T = 2 | 0.741 | 0.259 | 22.677.007 | Intron3 | 52 |
| 187 | rs12546464 | C = 1 | T = 2 | 0.25 | 0.75 | 22.683.416 | Intron3 | 53 |
| 188 | rs11135676 | C = 1 | T = 2 | 0.534 | 0.466 | 22.685.029 | Intron3 | 54 |
| 189 | rs7013223 | C = 1 | G = 2 | 0.638 | 0.362 | 22.689.809 | Intron3 | 55 |
| 190 | rs12676845 | A = 1 | G = 2 | 0.246 | 0.754 | 22.691.280 | Intron3 | 56 |
| 191 | rs4872011 | C = 1 | T = 2 | 0.554 | 0.446 | 22.691.521 | Intron3 | 57 |
| 192 | rs4872012 | C = 1 | T = 2 | 0.45 | 0.55 | 22.702.726 | Intron3 | 58 |
| 195 | rs6557594 | A = 1 | G = 2 | 0.161 | 0.839 | 22.711.133 | Intron3 | 59 |
| 196 | rs953561 | A = 1 | C = 2 | 0.533 | 0.467 | 22.717.805 | Intron3 | 60 |
| 197 | rs12679264 | C = 1 | T = 2 | 0.867 | 0.133 | 22.718.141 | Intron3 | 61 |
| 198 | rs11985147 | A = 1 | C = 2 | 0.592 | 0.408 | 22.720.743 | Intron3 | 62 |
| 199 | rs4872029 | C = 1 | T = 2 | 0.217 | 0.783 | 22.722.224 | Intron3 | 63 |
| 200 | rs12681784 | C = 1 | T = 2 | 0.183 | 0.817 | 22.723.584 | Intron3 | 64 |
| 201 | rs12682168 | G = 1 | T = 2 | 0.17 | 0.83 | 22.723.667 | Intron3 | 65 |
| 202 | rs1533308 | A = 1 | G = 2 | 0.625 | 0.375 | 22.725.338 | Intron3 | 66 |
| 203 | rs1996148 | A = 1 | G = 2 | 0.3 | 0.7 | 22.727.450 | Intron3 | 67 |
| 205 | rs12677017 | G = 1 | T = 2 | 0.542 | 0.458 | 22.732.080 | Intron2 | 68 |
| 206 | rs12386970 | A = 1 | G = 2 | 0.392 | 0.608 | 22.741.098 | Intron2 | 69 |
| 209 | rs11986200 | A = 1 | G = 2 | 0.45 | 0.55 | 22.754.154 | Intron2 | 70 |
| 210 | rs1040053 | A = 1 | C = 2 | 0.161 | 0.839 | 22.755.164 | Intron2 | 71 |
| 212 | rs12676524 | A = 1 | C = 2 | 0.664 | 0.336 | 22.759.502 | Intron2 | 72 |
| 215 | rs7816775 | C = 1 | T = 2 | 0.873 | 0.127 | 22.764.783 | Intron2 | 73 |
| 218 | rs7007235 | C = 1 | T = 2 | 0.775 | 0.225 | 22.768.957 | Intron2 | 74 |
| 220 | rs7005929 | C = 1 | T = 2 | 0.833 | 0.167 | 22.774.182 | Intron2 | 75 |
| 221 | rs9644059 | C = 1 | T = 2 | 0.307 | 0.693 | 22.775.383 | Intron2 | 76 |
| 226 | rs2466241 | A = 1 | C = 2 | 0.367 | 0.633 | 22.800.053 | Intron2 | 77 |
| 227 | rs2246578 | C = 1 | T = 2 | 0.5 | 0.5 | 22.800.509 | Intron2 | 78 |
| 229 | rs13270026 | A = 1 | G = 2 | 0.275 | 0.725 | 22.807.539 | Intron2 | 79 |
| 231 | rs4872037 | A = 1 | G = 2 | 0.142 | 0.858 | 22.816.375 | Intron2 | 80 |
| 237 | rs1009613 | C = 1 | T = 2 | 0.314 | 0.686 | 22.824.999 | Intron2 | 81 |
| 239 | rs17088762 | C = 1 | G = 2 | 0.2 | 0.8 | 22.826.176 | Intron2 | 82 |
| 240 | rs2457422 | C = 1 | T = 2 | 0.368 | 0.632 | 22.826.949 | Intron2 | 83 |
| 242 | rs11780160 | A = 1 | G = 2 | 0.167 | 0.833 | 22.829.247 | Intron2 | 84 |
| 246 | rs2457423 | C = 1 | T = 2 | 0.758 | 0.242 | 22.831.172 | Intron2 | 85 |
| 247 | rs2466214 | A = 1 | G = 2 | 0.8 | 0.2 | 22.831.274 | Intron2 | 86 |
| 248 | rs10503722 | C = 1 | T = 2 | 0.833 | 0.167 | 22.831.800 | Intron2 | 87 |
| 250 | rs2466219 | A = 1 | C = 2 | 0.625 | 0.375 | 22.834.591 | Intron2 | 88 |
| 251 | rs2466220 | A = 1 | C = 2 | 0.6 | 0.4 | 22.835.130 | Intron2 | 89 |
| 254 | rs11135687 | A = 1 | G = 2 | 0.15 | 0.85 | 22.840.680 | Intron2 | 90 |

Preferably the SNP is allele T of SNP225.

More preferably, said haplotype comprises or consists of several SNPs selected from the group consisting of SNP224, SNP225, SNP244, more particularly the following haplotype:

1-1-1 (i.e. SNP224 is T, SNP225 is T and SNP244 is A).

The invention further provides a method for preventing type 2 diabetes in a subject, more particularly a subject with obesity, comprising detecting the presence of an alteration in the PEBP4 gene locus in a sample from the subject, the presence of said alteration being indicative of the predisposition to type 2 diabetes, and administering a prophylactic treatment against type 2 diabetes.

The alteration may be determined at the level of the PEBP4 gDNA, RNA or polypeptide. Optionally, the detection is performed by sequencing all or part of the PEBP4 gene or by selective hybridisation or amplification of all or part of the PEBP4 gene. More preferably a PEBP4 gene specific amplification is carried out before the alteration identification step.

An alteration in the PEBP4 gene locus may be any form of mutation(s), deletion(s), rearrangement(s) and/or insertions in the coding and/or non-coding region of the locus, alone or in various combination(s). Mutations more specifically include point mutations. Deletions may encompass any region of two or more residues in a coding or non-coding portion of the gene locus, such as from two residues up to the entire gene or locus. Typical deletions affect smaller regions, such as domains (introns) or repeated sequences or fragments of less than about 50 consecutive base pairs, although larger deletions may occur as well. Insertions may encompass the addition of one or several residues in a coding or non-coding portion of the gene locus. Insertions may typically comprise an addition of between 1 and 50 base pairs in the gene locus. Rearrangement includes inversion of sequences. The PEBP4 gene locus alteration may result in the creation of stop codons, frameshift mutations, amino acid substitutions, particular RNA splicing or processing, product instability, truncated polypeptide production, etc. The alteration may result in the production of a PEBP4 polypeptide with altered function, stability, targeting or structure.

The alteration may also cause a reduction in protein expression or, alternatively, an increase in said production.

In a particular embodiment of the method according to the present invention, the alteration in the PEBP4 gene locus is selected from a point mutation, a deletion and an insertion in the PEBP4 gene or corresponding expression product, more preferably a point mutation and a deletion.

In any method according to the present invention, one or several SNP in the PEBP4 gene and certain haplotypes comprising SNP in the PEBP4 gene can be used in combination with other SNP or haplotype associated with type 2 diabetes and located in other gene(s).

In another variant, the method comprises detecting the presence of an altered PEBP4 RNA expression. Altered RNA expression includes the presence of an altered RNA sequence, the presence of an altered RNA splicing or processing, the presence of an altered quantity of RNA, etc. These may be detected by various techniques known in the art, including by sequencing all or part of the PEBP4 RNA or by selective hybridisation or selective amplification of all or part of said RNA, for instance.

In a further variant, the method comprises detecting the presence of an altered PEBP4 polypeptide expression. Altered PEBP4 polypeptide expression includes the presence of an altered polypeptide sequence, the presence of an altered quantity of PEBP4 polypeptide, the presence of an altered tissue distribution, etc. These may be detected by various techniques known in the art, including by sequencing and/or binding to specific ligands (such as antibodies), for instance.

As indicated above, various techniques known in the art may be used to detect or quantify altered PEBP4 gene or RNA expression or sequence, including sequencing, hybridisation, amplification and/or binding to specific ligands (such as antibodies). Other suitable methods include allele-specific oligonucleotide (ASO), allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, heteroduplex analysis, RNase protection, chemical mismatch cleavage, ELISA, radio-immunoassays (RIA) and immuno-enzymatic assays (IEMA).

Some of these approaches (e.g., SSCA and CGGE) are based on a change in electrophoretic mobility of the nucleic acids, as a result of the presence of an altered sequence. According to these techniques, the altered sequence is visualized by a shift in mobility on gels. The fragments may then be sequenced to confirm the alteration.

Some others are based on specific hybridisation between nucleic acids from the subject and a probe specific for wild type or altered PEBP4 gene or RNA. The probe may be in suspension or immobilized on a substrate. The probe is typically labeled to facilitate detection of hybrids.

Some of these approaches are particularly suited for assessing a polypeptide sequence or expression level, such as Northern blot, ELISA and RIA. These latter require the use of a ligand specific for the polypeptide, more preferably of a specific antibody.

In a particular, preferred, embodiment, the method comprises detecting the presence of an altered PEBP4 gene expression profile in a sample from the subject. As indicated above, this can be accomplished more preferably by sequencing, selective hybridisation and/or selective amplification of nucleic acids present in said sample.

### Sequencing

Sequencing can be carried out using techniques well known in the art, using automatic sequencers. The sequencing may be performed on the complete PEBP4 gene or, more preferably, on specific domains thereof, typically those known or suspected to carry deleterious mutations or other alterations.

### Amplification

Amplification is based on the formation of specific hybrids between complementary nucleic acid sequences that serve to initiate nucleic acid reproduction.

Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Preferred techniques use allele-specific PCR or PCR-SSCP. Amplification usually requires the use of specific nucleic acid primers, to initiate the reaction.

Nucleic acid primers useful for amplifying sequences from the PEBP4 gene or locus are able to specifically hybridize with a portion of the PEBP4 gene locus that flank a target region of said locus, said target region being altered in certain subjects having type 2 diabetes. Examples of such target regions are provided in Table 3A or Table 3B.

Primers that can be used to amplify PEBP4 target region comprising SNPs as identified in Tables 3A or 3B may be designed based on the sequence of SEQ ID No 1 or on the genomic sequence of PEBP4. In a particular embodiment, primers may be designed based on the sequence of SEQ ID Nos 3-90.

Typical primers of this invention are single-stranded nucleic acid molecules of about 5 to 60 nucleotides in length, more preferably of about 8 to about 25 nucleotides in length. The sequence can be derived directly from the sequence of the PEBP4 gene locus. Perfect complementarity is preferred, to ensure high specificity. However, certain mismatch may be tolerated.

The invention also concerns the use of a nucleic acid primer or a pair of nucleic acid primers as described above in a method of detecting the presence of or predisposition to type 2 diabetes in a subject, in particular in a subject with obesity.

### Selective Hybridization

Hybridization detection methods are based on the formation of specific hybrids between complementary nucleic acid sequences that serve to detect nucleic acid sequence alteration(s).

A particular detection technique involves the use of a nucleic acid probe specific for wild type or altered PEBP4 gene or RNA, followed by the detection of the presence of a hybrid. The probe may be in suspension or immobilized on a substrate or support (as in nucleic acid array or chips technologies). The probe is typically labeled to facilitate detection of hybrids.

In this regard, a particular embodiment of this invention comprises contacting the sample from the subject with a nucleic acid probe specific for an altered PEBP4 gene locus, and assessing the formation of an hybrid. In a particular, preferred embodiment, the method comprises contacting simultaneously the sample with a set of probes that are specific, respectively, for wild type PEBP4 gene locus and for various altered forms thereof. In this embodiment, it is possible to detect directly the presence of various forms of alterations in the PEBP4 gene locus in the sample. Also, various samples from various subjects may be treated in parallel.

Within the context of this invention, a probe refers to a polynucleotide sequence which is complementary to and capable of specific hybridisation with a (target portion of a) PEBP4 gene or RNA, and which is suitable for detecting polynucleotide polymorphisms associated with PEBP4 alleles which predispose to or are associated with obesity or an associated disorder. Probes are preferably perfectly complementary to the PEBP4 gene, RNA, or target portion thereof. Probes typically comprise single-stranded nucleic acids of between 8 to 1000 nucleotides in length, for instance of between 10 and 800, more preferably of between 15 and 700, typically of between 20 and 500. It should be understood that longer probes may be used as well. A preferred probe of this invention is a single stranded nucleic acid molecule of between 8 to 500 nucleotides in length, which can specifically hybridise to a region of a PEBP4 gene or RNA that carries an alteration.

A specific embodiment of this invention is a nucleic acid probe specific for an altered (e.g., a mutated) PEBP4 gene or RNA, i.e., a nucleic acid probe that specifically hybridises to said altered PEBP4 gene or RNA and essentially does not hybridise to a PEBP4 gene or RNA lacking said alteration. Specificity indicates that hybridisation to the target sequence generates a specific signal which can be distinguished from the signal generated through non-specific hybridisation. Perfectly complementary sequences are preferred to design probes according to this invention. It should be understood, however, that a certain degree of mismatch may be tolerated, as long as the specific signal may be distinguished from non-specific hybridisation.

Particular examples of such probes are nucleic acid sequences complementary to a target portion of the genomic region including the PEBP4 gene or RNA carrying a point mutation as listed in Table 3A or Table 3B above. More particularly, the probes can comprise a sequence selected from the group consisting of SEQ ID Nos 3-90 or a fragment thereof comprising the SNP or a complementary sequence thereof.

The sequence of the probes can be derived from the sequences of the PEBP4 gene and RNA as provided in the present application. Nucleotide substitutions may be performed, as well as chemical modifications of the probe. Such chemical modifications may be accomplished to increase the stability of hybrids (e.g., intercalating groups) or to label the probe. Typical examples of labels include, without limitation, radioactivity, fluorescence, luminescence, enzymatic labeling, etc.

The invention also concerns the use of a nucleic acid probe as described above in a method of detecting the presence of or predisposition to type 2 diabetes in a subject or in a method of assessing the response of a subject to a treatment of type 2 diabetes or an associated disorder.

### Specific Ligand Binding

As indicated above, alteration in the PEBP4 gene locus may also be detected by screening for alteration(s) in PEBP4 polypeptide sequence or expression levels. In this regard, a specific embodiment of this invention comprises contacting the sample with a ligand specific for a PEBP4 polypeptide and determining the formation of a complex.

Different types of ligands may be used, such as specific antibodies. In a specific embodiment, the sample is contacted with an antibody specific for a PEBP4 polypeptide and the formation of an immune complex is determined. Various methods for detecting an immune complex can be used, such as ELISA, radioimmunoassays (RIA) and immuno-enzymatic assays (IEMA).

Within the context of this invention, an antibody designates a polyclonal antibody, a monoclonal antibody, as well as fragments or derivatives thereof having substantially the same antigen specificity. Fragments include Fab, Fab′2, CDR regions, etc. Derivatives include single-chain antibodies, humanized antibodies, poly-functional antibodies, etc.

An antibody specific for a PEBP4 polypeptide designates an antibody that selectively binds a PEBP4 polypeptide, namely, an antibody raised against a PEBP4 polypeptide or an epitope-containing fragment thereof. Although non-specific binding towards other antigens may occur, binding to the target PEBP4 polypeptide occurs with a higher affinity and can be reliably discriminated from non-specific binding.

In a specific embodiment, the method comprises contacting a sample from the subject with (a support coated with) an antibody specific for an altered form of a PEBP4 polypeptide, and determining the presence of an immune complex. In a particular embodiment, the sample may be contacted simultaneously, or in parallel, or sequentially, with various (supports coated with) antibodies specific for different forms of a PEBP4 polypeptide, such as a wild type and various altered forms thereof.

The invention also concerns the use of a ligand, preferably an antibody, a fragment or a derivative thereof as described above, in a method of detecting the presence of or predisposition to type 2 diabetes in a subject, in particular in a subject with obesity.

In order to carry out the methods of the invention, one can employ diagnostic kits comprising products and reagents for detecting in a sample from a subject the presence of an alteration in the PEBP4 gene or polypeptide, in the PEBP4 gene or polypeptide expression, and/or in PEBP4 activity. Said diagnostic kit comprises any primer, any pair of primers, any nucleic acid probe and/or any ligand, preferably antibody, described in the present invention. Said diagnostic kit can further comprise reagents and/or protocols for performing a hybridization, amplification or antigen-antibody immune reaction.

The diagnosis methods can be performed in vitro, ex vivo or in vivo, preferably in vitro or ex vivo. They use a sample from the subject, to assess the status of the PEBP4 gene locus. The sample may be any biological sample derived from a subject, which contains nucleic acids or polypeptides. Examples of such samples include fluids, tissues, cell samples, organs, biopsies, etc. Most preferred samples are blood, plasma, saliva, urine, seminal fluid, etc. The sample may be collected according to conventional techniques and used directly for diagnosis or stored. The sample may be treated prior to performing the method, in order to render or improve availability of nucleic acids or polypeptides for testing. Treatments include, for instant, lysis (e.g., mechanical, physical, chemical, etc.), centrifugation, etc. Also, the nucleic acids and/or polypeptides may be pre-purified or enriched by conventional techniques, and/or reduced in complexity. Nucleic acids and polypeptides may also be treated with enzymes or other chemical or physical treatments to produce fragments thereof. Considering the high sensitivity of the claimed methods, very few amounts of sample are sufficient to perform the assay.

As indicated, the sample is preferably contacted with reagents such as probes, primers or ligands in order to assess the presence of an altered PEBP4 gene locus. Contacting may be performed in any suitable device, such as a plate, tube, well, glass, etc. In specific embodiments, the contacting is performed on a substrate coated with the reagent, such as a nucleic acid array or a specific ligand array. The substrate may be a solid or semi-solid substrate such as any support comprising glass, plastic, nylon, paper, metal, polymers and the like. The substrate may be of various forms and sizes, such as a slide, a membrane, a bead, a column, a gel, etc. The contacting may be made under any condition suitable for a complex to be formed between the reagent and the nucleic acids or polypeptides of the sample.

The finding of an altered PEBP4 polypeptide, RNA or DNA in the sample is indicative of the presence of an altered PEBP4 gene locus in the subject, which can be correlated to the presence, predisposition or stage of progression of type 2 diabetes. For example, an individual having a germ line PEBP4 mutation has an increased risk of developing type 2 diabetes. The determination of the presence of an altered PEBP4 gene locus in a subject also allows the design of appropriate therapeutic intervention, which is more effective and customized.

### Linkage Disequilibrium

Once a first SNP has been identified in a genomic region of interest, more particularly in PEBP4 gene locus, the practitioner of ordinary skill in the art can easily identify additional SNPs in linkage disequilibrium with this first SNP. Indeed, any SNP in linkage disequilibrium with a first SNP associated with type 2 diabetes will be associated with this trait. Therefore, once the association has been demonstrated between a given SNP and type 2 diabetes, the discovery of additional SNPs associated with this trait can be of great interest in order to increase the density of SNPs in this particular region.

Identification of additional SNPs in linkage disequilibrium with a given SNP involves: (a) amplifying a fragment from the genomic region comprising or surrounding a first SNP from a plurality of individuals; (b) identifying of second SNPs in the genomic region harboring or surrounding said first SNP; (c) conducting a linkage disequilibrium analysis between said first SNP and second SNPs; and (d) selecting said second SNPs as being in linkage disequilibrium with said first marker. Subcombinations comprising steps (b) and (c) are also contemplated.

Methods to identify SNPs and to conduct linkage disequilibrium analysis can be carried out by the skilled person without undue experimentation by using well-known methods.

These SNPs in linkage disequilibrium can also be used in the methods according to the present invention, and more particularly in the diagnosic methods according to the present invention.

For example, a linkage locus of Crohn's disease has been mapped to a large region spanning 18cM on chromosome 5q31 (Rioux et al., 2000 and 2001). Using dense maps of microsatellite markers and SNPs across the entire region, strong evidence of linkage disequilibrium (LD) was found. Having found evidence of LD, the authors developed an ultra-high-density SNP map and studied a denser collection of markers selected from this map. Multilocus analyses defined a single common risk haplotype characterised by multiple SNPs that were each independently associated using TDT. These SNPs were unique to the risk haplotype and essentially identical in their information content by virtue of being in nearly complete LD with one another. The equivalent properties of these SNPs make it impossible to identify the causal mutation within this region on the basis of genetic evidence alone.

### Causal Mutation

Mutations in the PEBP4 gene which are responsible for type 2 diabetes may be identified by comparing the sequences of the PEBP4 gene from patients presenting type 2 diabetes and control individuals. Based on the identified association of SNPs of PEBP4 and type 2 diabetes, the identified locus can be scanned for mutations. In a preferred embodiment, functional regions such as exons and splice sites, promoters and other regulatory regions of the PEBP4 gene are scanned for mutations. Preferably, patients presenting type 2 diabetes carry the mutation shown to be associated with type 2 diabetes and controls individuals do not carry the mutation or allele associated with type 2 diabetes or an associated disorder. It might also be possible that patients presenting type 2 diabetes carry the mutation shown to be associated with type 2 diabetes with a higher frequency than controls individuals.

The method used to detect such mutations generally comprises the following steps: amplification of a region of the PEBP4 gene comprising a SNP or a group of SNPs associated with type 2 diabetes from DNA samples of the PEBP4 gene from patients presenting type 2 diabetes and control individuals; sequencing of the amplified region; comparison of DNA sequences of the PEBP4 gene from patients presenting type 2 diabetes and control individuals; determination of mutations specific to patients presenting type 2 diabetes.

Therefore, identification of a causal mutation in the PEBP4 gene can be carried out by the skilled person without undue experimentation by using well-known methods.

For example, the causal mutations have been identified in the following examples by using routine methods.

Hugot et al. (2001) applied a positional cloning strategy to identify gene variants with susceptibly to Crohn's disease in a region of chromosome 16 previously found to be linked to susceptibility to Crohn's disease. To refine the location of the potential susceptibility locus 26 microsatellite markers were genotyped and tested for association to Crohn's disease using the transmission disequilibrium test. A borderline significant association was found between one allele of the microsatellite marker D16S136. Eleven additional SNPs were selected from surrounding regions and several SNPs showed significant association. SNP5-8 from this region were found to be present in a single exon of the NOD2/CARD15 gene and shown to be non-synonymous variants. This prompted the authors to sequence the complete coding sequence of this gene in 50 CD patients. Two additional non-synonymous mutations (SNP12 and SNP13) were found. SNP13 was most significant associated (p=6×10⁻⁶) using the pedigree transmission disequilibrium test. In another independent study, the same variant was found also by sequencing the coding region of this gene from 12 affected individuals compared to 4 controls (Ogura et al., 2001). The rare allele of SNP13 corresponded to a 1-bp insertion predicted to truncate the NOD2/CARD15 protein.

This allele was also present in normal healthy individuals, albeit with significantly lower frequency as compared to the controls.

Similarly, Lesage et al. (2002) performed a mutational analyses of CARD15 in 453 patients with CD, including 166 sporadic and 287 familial cases, 159 patients with ulcerative colitis (UC), and 103 healthy control subjects by systematic sequencing of the coding region. Of 67 sequence variations identified, 9 had an allele frequency >5% in patients with CD. Six of them were considered to be polymorphisms, and three (SNP12-R702W, SNP8-G908R, and SNP13-1007fs) were confirmed to be independently associated with susceptibility to CD. Also considered as potential disease-causing mutations (DCMs) were 27 rare additional mutations. The three main variants (R702W, G908R, and 1007fs) represented 32%, 18%, and 31%, respectively, of the total CD mutations, whereas the total of the 27 rare mutations represented 19% of DCMs. Altogether, 93% of the mutations were located in the distal third of the gene. No mutations were found to be associated with UC. In contrast, 50% of patients with CD carried at least one DCM, including 17% who had a double mutation.

The present invention demonstrates the correlation between type 2 diabetes and the PEBP4 gene locus. The invention thus provides a novel target of therapeutic intervention. Various approaches can be contemplated to restore or modulate the PEBP4 activity or function in a subject, particularly those carrying an altered PEBP4 gene locus. Supplying wild-type function to such subjects is expected to suppress phenotypic expression of type 2 diabetes in a pathological cell or organism. The supply of such function can be accomplished through gene or protein therapy, or by administering compounds that modulate or mimic PEBP4 polypeptide activity (e.g., agonists as identified in the above screening assays).

Other molecules with PEBP4 activity (e.g., peptides, drugs, PEBP4 agonists, or organic compounds) may also be used to restore functional PEBP4 activity in a subject or to suppress the deleterious phenotype in a cell.

Restoration of functional PEBP4 gene function in a cell may be used to prevent the development of type 2 diabetes or to reduce progression of said diseases. Such a treatment may suppress the type 2 diabetes-associated phenotype of a cell, particularly those cells carrying a deleterious allele.

Further aspects and advantages of the present invention will be disclosed in the following experimental section, which should be regarded as illustrative and not limiting the scope of the present application.

### EXAMPLES

### 1. GenomeHIP Platform to Identify the Chromosome 8 Susceptibility Gene

The GenomeHIP platform was applied to allow rapid identification of a TYPE 2 DIABETES susceptibility gene.

Briefly, the technology consists of forming pairs from the DNA of related individuals. Each DNA is marked with a specific label allowing its identification. Hybrids are then formed between the two DNAs. A particular process (WO00/53802) is then applied that selects all fragments identical-by-descent (IBD) from the two DNAs in a multi step procedure. The remaining IBD enriched DNA is then scored against a BAC clone derived DNA microarray that allows the positioning of the IBD fraction on a chromosome.

The application of this process over many different families results in a matrix of IBD fractions for each pair from each family. Statistical analyses then calculate the minimal IBD regions that are shared between all families tested. Significant results (p-values) are evidence for linkage of the positive region with the trait of interest (here TYPE 2 DIABETES). The linked interval can be delimited by the two most distant clones showing significant p-values.

In the present study, 119 diabetes (type 2 diabetes) relative pairs, were submitted to the GenomeHIP process. The resulting IBD enriched DNA fractions were then labelled with Cy5 fluorescent dyes and hybridised against a DNA array consisting of 2263 BAC clones covering the whole human genome with an average spacing of 1.2 Mega base pairs. Non-selected DNA labelled with Cy3 was used to normalize the signal values and compute ratios for each clone. Clustering of the ratio results was then performed to determine the IBD status for each clone and pair.

By applying this procedure, several BAC clones spanning approximately 4.5 Mega bases in the region on chromosome 8 were identified, that showed significant evidence for linkage to type 2 diabetes (p=1.90E-10).

### 2. Identification of an TYPE 2 DIABETES Susceptibility Gene on Chromosome 8

By screening the aforementioned 5.8 Megabases in the linked chromosomal region, the inventors identified the PEBP4 gene as a candidate for type 2 diabetes. This gene is indeed present in the critical interval, with evidence for linkage delimited by the clones outlined above.

TABLE 4
Linkage results for chromosome 8 in the PEBP4 locus: Indicated is the region
correspondent to BAC clones with evidence for linkage. The start and stop positions of the
clones correspond to their genomic location based on NCBI Build 35 sequence respective
to the start of the chromosome (p-ter).

| | Clone | | | % of | IBD | |
|---|---|---|---|---|---|---|
| Human | IG-Name | | | informative | sharing | |
| chrom. | (Origin name) | Start | Stop | pairs | (%) | p-value |
| 8 | BACA12ZD05 | 17.513.477 | 17.685.793 | 60% | 0.83 | 7.1 * 10⁻² |
| | (RP11-499D5) | | | | | |
| 8 | BACA1ZA04 | 19.416.907 | 19.417.225 | 76% | 0.86 | 1.1 * 10⁻² |
| | (RP11-51C1) | | | | | |
| 8 | BACA12ZD06 | 20.134.018 | 20.300.107 | 63% | 0.95 | 7.6 * 10⁻⁶ |
| | (RP11-399K16) | | | | | |
| 8 | BACA12ZC07 | 21.982.444 | 22.152.133 | 99% | 0.97 | 1.9 * 10⁻¹⁰ |
| | (RP11-515L12) | | | | | |
| 8 | BACA12ZD02 | 23.245.195 | 23.521.961 | 92% | 0.91 | 2.0 * 10⁻⁵ |
| | (RP11-304K15) | | | | | |
| 8 | PADA9ZE02 | 25.245.630 | 25.406.418 | 99% | 0.82 | 4.1 * 10⁻² |
| | (RP11-76B12) | | | | | |
| 8 | BACA4ZD02 | 26.308.669 | 26.476.264 | 64% | 0.79 | 2.6 * 10⁻¹ |
| | (none) | | | | | |

Taken together, the linkage results provided in the present application, identifying the human PEBP4 gene in the critical interval of genetic alterations linked to TYPE 2 DIABETES on chromosome 8.

### 3. Association Study

### Single SNP and Haplotype Analysis:

Differences in allele distributions between cases and controls were screened for all SNPs. Three cases and controls sample have been used in the analysis:
Sample I corresponding on 1034 TYPE 2 DIABETES cases versus 1034 normo-glycemic controles;
Sample II corresponding on 732 TYPE 2 DIABETES with BMI 27 versus 678 normo-glycemic Controls with BMI<27;

Association analyses have been conducted using COCAPHASE v2.404 software from the UNPHASED suite of programs.

The method is based on likelihood ratio tests in a logistic model:

$$log ( \frac{p}{1 - p} ) = mu + {\sum }_{i} {beta}_{i} \cdot {x}_{i}$$

where p is the probability of a chromosome being a “case” rather than a “control”, xᵢ are variables which represent the allele or haplotypes in some way depending upon the particular test, and mu and betaᵢ are coefficients to be estimated. Reference for this application of log-linear models is Cordell & Clayton, AJHG (2002)

In cases of uncertain haplotype, the method for case-control sample is a standard unconditional logistic regression identical to the model-free method T5 of EHPLUS (Zhao et al Hum Hered (2000) and the log-linear modelling of Mander. The betaᵢ are log odds ratios for the haplotypes. The EM algorithm is used to obtain maximum likelihood frequency estimates.

### SNP Genotype Analysis:

Differences in genotype distributions between cases and controls were screened for all SNPs. For each SNPs, three genotype is possible genotype RR, genotype Rn and genotype nn where R represented the associate allele of the SNP with TYPE 2 DIABETES. Dominant transmission model for associated risk allele (R) vs the non-risk allele (n) were tested by counting n Ra and R R genotype together. The statistic test was carried out using the standard Chi-square independence test with 1 df (genotype distribution, 2×2 table). Recessive transmission model for associated allele (R) were tested by counting the non-risk nn and nR genotypes together. The statistic test was carried out using the standard Chi-square independence test with 1 df (genotype distribution, 2×2 table). Additive transmission model for associated allele (a) were tested using the standard Chi-square independence test with 2 df (genotype distribution, 2×3 table).

3.1—Association with Single SNPs, Allele Distribution Statistics Test:

TABLE 4
1034 Diabetes versus 1034 normo-glycemic Controls: sample I

| SNP | dbSNP | | | Frequence | | Frequence | Risk | |
|---|---|---|---|---|---|---|---|---|
| identity | reference | Allele | Cases | in Cases | Controls | in Controls | Allelel | p-values |
| 214 | rs11779201 | 1 | 417 | 0.20 | 365 | 0.18 | A | 0.040190 |
| | | 2 | 1641 | 0.80 | 1691 | 0.82 | | |
| 217 | rs4284046 | 1 | 376 | 0.18 | 311 | 0.15 | A | 0.006016 |
| | | 2 | 1678 | 0.82 | 1747 | 0.85 | | |
| 223 | rs11785590 | 1 | 402 | 0.20 | 464 | 0.23 | | |
| | | 2 | 1656 | 0.80 | 1578 | 0.77 | T | 0.012340 |
| 224 | rs937969 | 1 | 1250 | 0.61 | 1330 | 0.65 | | |
| | | 2 | 804 | 0.39 | 720 | 0.35 | T | 0.007668 |
| 225 | rs11784354 | 1 | 1496 | 0.74 | 1568 | 0.77 | | |
| | | 2 | 534 | 0.26 | 456 | 0.23 | T | 0.005124 |
| 228 | rs2466180 | 1 | 1718 | 0.84 | 1769 | 0.86 | | |
| | | 2 | 338 | 0.16 | 285 | 0.14 | T | 0.021830 |
| 230 | rs11135684 | 1 | 381 | 0.19 | 325 | 0.16 | A | |
| | | 2 | 1677 | 0.81 | 1735 | 0.84 | | 0.019780 |
| 232 | rs17088737 | 1 | 364 | 0.18 | 426 | 0.21 | | |
| | | 2 | 1690 | 0.82 | 1630 | 0.79 | T | 0.014680 |
| 244 | rs2466213 | 1 | 1202 | 0.59 | 1134 | 0.55 | A | |
| | | 2 | 852 | 0.41 | 918 | 0.45 | | 0.035100 |
| 245 | rs11775299 | 1 | 1452 | 0.71 | 1379 | 0.67 | C | |
| | | 2 | 606 | 0.29 | 667 | 0.33 | | 0.028930 |
| 255 | rs2457426 | 1 | 1187 | 0.58 | 1106 | 0.54 | A | |
| | | 2 | 863 | 0.42 | 944 | 0.46 | | 0.010820 |

TABLE 5
732 Diabetes with BMT ≧ 27 vs. 678 normo-glycemic Controls with BMT <
27: sample II

| SNP | dbSNP | | | Frequence | | Frequence | Risk | |
|---|---|---|---|---|---|---|---|---|
| identity | reference | Allele | Cases | in Cases | Controls | in Controls | Allele | p-values |
| 184 | rs7846693 | 1 | 1061 | 0.77 | 921 | 0.73 | C | 0.01719 |
| | | 2 | 319 | 0.23 | 343 | 0.27 | | |
| 193 | rs17088624 | 1 | 624 | 0.43 | 511 | 0.38 | A | |
| | | 2 | 830 | 0.57 | 831 | 0.62 | | 0.009209 |
| 196 | rs953561 | 1 | 849 | 0.58 | 840 | 0.63 | A | 0.02277 |
| | | 2 | 603 | 0.42 | 500 | 0.37 | | |
| 204 | rs10503720 | 1 | 1189 | 0.82 | 1145 | 0.85 | | |
| | | 2 | 267 | 0.18 | 205 | 0.15 | T | 0.02547 |
| 208 | rs2063688 | 1 | 297 | 0.20 | 228 | 0.17 | | |
| | | 2 | 1159 | 0.80 | 1118 | 0.83 | | 0.01887 |
| 211 | rs11135681 | 1 | 623 | 0.43 | 521 | 0.39 | C | 0.0423 |
| | | 2 | 827 | 0.57 | 809 | 0.61 | | |
| 214 | rs11779201 | 1 | 299 | 0.21 | 224 | 0.17 | A | 0.007648 |
| | | 2 | 1159 | 0.79 | 1126 | 0.83 | | |
| 217 | rs4284046 | 1 | 271 | 0.19 | 187 | 0.14 | A | 0.0005904 |
| | | 2 | 1183 | 0.81 | 1163 | 0.86 | | |
| 219 | rs11777000 | 1 | 592 | 0.41 | 607 | 0.45 | | |
| | | 2 | 866 | 0.59 | 739 | 0.55 | C | 0.01628 |
| 222 | rs2457436 | 1 | 472 | 0.33 | 387 | 0.29 | A | 0.02935 |
| | | 2 | 980 | 0.67 | 961 | 0.71 | | |
| 223 | rs11785590 | 1 | 299 | 0.18 | 313 | 0.23 | | |
| | | 2 | 1192 | 0.82 | 1025 | 0.77 | T | 0.0007923 |
| 224 | rs937969 | 1 | 877 | 0.60 | 887 | 0.66 | | |
| | | 2 | 577 | 0.40 | 457 | 0.34 | T | 0.001854 |
| 225 | rs11784354 | 1 | 1050 | 0.73 | 1061 | 0.79 | | |
| | | 2 | 390 | 0.27 | 277 | 0.21 | T | 8.04E−05 |
| 228 | rs2466180 | 1 | 1211 | 0.83 | 1176 | 0.87 | | |
| | | 2 | 245 | 0.17 | 170 | 0.13 | T | 0.001724 |
| 230 | rs11135684 | 1 | 273 | 0.19 | 199 | 0.15 | A | 0.004704 |
| | | 2 | 1185 | 0.81 | 1151 | 0.85 | | |
| 232 | rs17088737 | 1 | 251 | 0.17 | 277 | 0.21 | | |
| | | 2 | 1203 | 0.83 | 1073 | 0.79 | T | 0.02762 |
| 244 | rs2466213 | 1 | 855 | 0.59 | 734 | 0.54 | A | 0.02017 |
| | | 2 | 599 | 0.41 | 614 | 0.46 | | |
| 245 | rs11775299 | 1 | 1039 | 0.71 | 903 | 0.67 | C | 0.01951 |
| | | 2 | 419 | 0.29 | 441 | 0.33 | | |
| 255 | rs2457426 | 1 | 851 | 0.59 | 722 | 0.54 | A | 0.008142 |
| | | 2 | 599 | 0.41 | 622 | 0.46 | | |

3.2—Association with Single SNPs, Genotype Distributions Statistics Test:
a—1035 Diabetes Versus 1035 Normo-Glycemic Controls:

DOMINENT Model for RISK allele (R) vs non-risk allele (n):

| | | | Geno- | | | |
|---|---|---|---|---|---|---|
| | | | type | Geno- | Yates | |
| SNP | dbSNP | | RR + | type | Statistic | |
| identity | reference | Sample | Rn | nn | (df = 1) | p-values |
| 204 | rs10503720 | cases | 353 | 675 | 5.07 | 0.024340 |
| | | controls | 305 | 725 | | |
| 208 | rs2063688 | cases | 381 | 647 | 5.09 | 0.024100 |
| | | controls | 331 | 696 | | |
| 214 | rs11779201 | cases | 381 | 648 | 6.2 | 0.012740 |
| | | controls | 326 | 702 | | |
| 217 | rs4284046 | cases | 349 | 678 | 9.84 | 0.001710 |
| | | controls | 283 | 746 | | |
| 223 | rs11785590 | cases | 991 | 38 | 5.32 | 0.021090 |
| | | controls | 960 | 61 | | |
| 224 | rs937969 | cases | 646 | 381 | 5.89 | 0.015250 |
| | | controls | 590 | 435 | | |
| 225 | rs11784354 | cases | 473 | 542 | 8.94 | 0.002790 |
| | | controls | 404 | 608 | | |
| 228 | rs2466180 | cases | 319 | 709 | 6.39 | 0.011470 |
| | | controls | 266 | 761 | | |
| 230 | rs11135684 | cases | 356 | 673 | 7.1 | 0.007700 |
| | | controls | 299 | 731 | | |
| 232 | rs17088737 | cases | 998 | 29 | 5.71 | 0.016820 |
| | | controls | 977 | 51 | | |
| 238 | rs7010513 | cases | 960 | 67 | 5.25 | 0.021890 |
| | | controls | 930 | 96 | | |
| 241 | rs17757261 | cases | 984 | 42 | 4.01 | 0.045100 |
| | | controls | 963 | 63 | | |
| 244 | rs2466213 | cases | 858 | 169 | 4.5 | 0.033860 |
| | | controls | 819 | 207 | | |
| 245 | rs11775299 | cases | 951 | 78 | 6.89 | 0.008850 |
| | | controls | 910 | 113 | | |
| 253 | rs17088800 | cases | 577 | 449 | 4.34 | 0.037170 |
| | | controls | 530 | 498 | | |
| 255 | rs11135688 | cases | 998 | 15 | 4.51 | 0.033670 |
| | | controls | 979 | 30 | | |

b—732 Diabetes with BMI≧27 Vs. 678 Normo-Glycemic Controls with BMI<27:

DOMINENT Model for RISK allele (R) vs non-risk allele (n)::

| | | | Geno- | | | |
|---|---|---|---|---|---|---|
| | | | type | Geno- | Yates | |
| SNP | dbSNP | | RR + | type | Statistic | |
| identity | reference | Sample | rn | nn | (df = 1) | p-values |
| 181 | Rs11781835 | cases | 440 | 288 | 3.89 | 0.048560 |
| | | controls | 442 | 231 | | |
| 193 | Rs17088624 | cases | 491 | 236 | 4.96 | 0.025960 |
| | | controls | 414 | 257 | | |
| 194 | Rs10102337 | cases | 445 | 284 | 4.14 | 0.041840 |
| | | controls | 447 | 226 | | |
| 204 | rs10503720 | cases | 250 | 478 | 6.89 | 0.008680 |
| | | controls | 187 | 488 | | |
| 208 | rs2063688 | cases | 272 | 456 | 6.8 | 0.009120 |
| | | controls | 206 | 467 | | |
| 211 | Rs11135681 | cases | 593 | 132 | 4.59 | 0.032220 |
| | | controls | 573 | 92 | | |
| 214 | rs11779201 | cases | 273 | 456 | 9.24 | 0.002360 |
| | | controls | 200 | 475 | | |
| 217 | rs4284046 | cases | 252 | 475 | 13.55 | 0.000230 |
| | | controls | 172 | 503 | | |
| 219 | Rs11777000 | cases | 463 | 266 | 5.43 | 0.019770 |
| | | controls | 468 | 205 | | |
| 223 | Rs11785590 | cases | 707 | 22 | 5.95 | 0.014690 |
| | | controls | 630 | 39 | | |
| 224 | Rs937969 | cases | 462 | 265 | 6.85 | 0.008840 |
| | | controls | 380 | 292 | | |
| 225 | Rs11784354 | cases | 350 | 370 | 16.47 | 0.00005 |
| | | controls | 252 | 417 | | |
| 228 | Rs2466180 | cases | 232 | 496 | 11.4 | 0.000730 |
| | | controls | 159 | 514 | | |
| 230 | Rs11135684 | cases | 255 | 474 | 10.14 | 0.001450 |
| | | controls | 182 | 493 | | |
| 234 | Rs13349266 | cases | 628 | 99 | 3.94 | 0.047090 |
| | | controls | 557 | 119 | | |
| 235 | Rs2466208 | cases | 594 | 133 | 4.08 | 0.043290 |
| | | controls | 518 | 153 | | |
| 238 | Rs7010513 | cases | 683 | 44 | 5 | 0.025370 |
| | | controls | 609 | 63 | | |
| 244 | Rs2466213 | cases | 611 | 116 | 5.11 | 0.023740 |
| | | controls | 534 | 140 | | |
| 245 | Rs11775299 | cases | 678 | 51 | 7.38 | 0.006600 |
| | | controls | 596 | 76 | | |
| 255 | Rs11135688 | cases | 706 | 11 | 3.91 | 0.047960 |
| | | controls | 644 | 22 | | |

3.3—Association with Haplotypes:

| | | | Frequency | Frequency | |
|---|---|---|---|---|---|
| | | Alleles | of | of | |
| | SNP used in | composing | haplotype | haplotype | |
| Sample | haplotype | haplotype | in cases | in controls | p-value |
| SAM- | 225-244 | 2-1 | 0.1794 | 0.1375 | 0.000527 |
| PLE I | | | | | |
| SAM- | 225-244 | 2-1 | 0.18 | 0.1263 | 3.13 * 10−5 |
| PLE II | | | | | |
| SAM- | 224-225-244 | 2-2-1 | 0.1716 | 0.1281 | 0.000226 |
| PLE I | | | | | |
| SAM- | 224-225-244 | 2-2-1 | 0.1714 | 0.1215 | 5.44 * 10−5 |
| PLE II | | | | | |

### REFERENCES

America Diabetes Association. 2003. Report of the Expert committee on the Diagnosis and Classification of Diabetes Mellitus. Diabetes Care 26:S5-S20.
Bell G I, Xiang K, Newman M V, Wu S, wright L G, Fajans S S, Spielman R S, Cox N J. 1991. Gene for non-insulino-dependent diabetes mellitus (maturity-onset diabetes of the young subtype) is linked to DNA polymorphism on human chromosome 20q. Proc Natl Acad Sci 88:1484-1488.
Byrne M M, Sturis J, Menzel S, Yamagata K, Fajans S S, Dronsfield M J, Bain S C, Hattersley A T, Velho G, Frogel P, Bell G I, Polonsky K S. 1996. Altered insulin secretory response to glucose in diabetic and nondiabetic subjects with mutations in the diabetes susceptibility gene MODY3 on chromosome 12. Diabetes 45:1503-1510.
Clement K, Pueyo M E, Vaxillaire M, Rakotoambinina B, Thuillier F, Passa P, Froguel P, Roberts J, Velho G. 1996. Assessment of insulin sensitivity in glucokinase-deficient subjects. Diabetologia 39: 82-90.
Cordell H J, Clayton D G. (2002) A unified stepwise regression procedure for evaluating the relative effects of polymorphisms within a gene using case/control or family data: application to HLA in type 1 diabetes. Am J Hum Genet. 70(1):124-41.
Frogel P, Vaxillaire M, Sun F, Velho G, Zouali H, Butel M O, Lesage S, Vionnet N, Clement K, Fougerousse F, et all. 1992. Close linkage of glucokinase locus on chromosome 7p to early-onset non-insulin-dependent diabetes mellitus. Nature 356: 162-164
Herman W H, Fajans S S, Oritz F J, Smith M J, Sturis J, Bell G I, Polonsky K S, Halter J B. 1994. Abnormal insulin secretion, not insulin resistance, is the genetic or primary defect of MODY in the RW pedigree. Diabetes 43: 40-46.
Hugot J P, Chamaillard M, Zouali H et al. (2001) Association of NOD2 leucine-rich repeat variants with susceptibility to Crohn's disease. Nature 411(6837):599-603.
Kadowaki T, Kadowaki H, Mori Y, To be K, Sakuta R, Suzuki Y, Tanabe Y, Sakura H, Awata T, Goto Y et all. 1994. A subtype of diabetes mellitus associated with a mutation of mitochondrial DNA. N Engl J Med 330: 962-968.
Khan C R, Flier J S, Bar R S, Archer J A, Gorden P, Martin M M, Roth J. 1976. The syndromes of insulin resistance and acanthosis nigricans. N Engl J Med 294: 739-745.
Knowler W C, Barrett-Connor E, Fowler S E, Hamman R F, Lachin J M, Walker E A, Nathan D M; Diabetes Prevention Program Research Group. 2002. Reduction in the incidence of diabetes with lifestyle intervention or metformin. N Engl J Med 346:393-403
Lesage S, Zouali H, Cezard Jp et al. (2002) CARD 15/NOD2 mutational analysis and genotype-phenotype correlation in 612 patients with inflammatory bowel disease. Am J Hum Genet. 70(4):845-857.
Ogura Y, Bonen D K, Inohara N (2001) A framshift mutation in NOD2 associated with susceptibility to Crohn's disease. Nature 411(6837):603-606.
Reardon W, Ross R J M, Sweeney M G, Luxon L M, Pembrey M E, Harding A E, Trembath R C. 1992. Diabetes mellitus associated with a pathogenic point mutation in mitochondrial DNA, Lancet 340:1376-1379.
Rioux J D, Daly M J, Silverberg M S et al. (2001) Genetic variation in the 5q31 cytokine gene cluster confers susceptibility to Crohn disease. Nat Genet 29(2): 223-228.
Rioux J D, Silverberg M S, Daly M J (2000) Genomewide search in Canadian families with inflammatory bowel disease reveals two novel susceptibility loci. Am J Hum Genet 66(6):1863-1870.
Taylor S I. 1992. Lilly Lecture: molecular machanisms of insulin resistance: lessons from patients with mutations in the insulin-receptor gene. Dibates 41:1473-1490.
Van den Ouwenland J M W, Lemkes H H P J, Ruitenbeek W, Sandkuijl L A, de Vijlder M F, Struyvenberg P A A, van de Kamp, Maassen J A. 1992. Mutation in mitochondrial tRNA (Leu(URR) gene in a large pedigree with maternally transmitted type II diabetes mellitus and deafiiess. Nature Genet 1:368-371.
Vaxillaire M, Boccio V, Philippi A, Vigouroux C, Terwilliger J, Passa P, Beckman J S, Velho G, Lathrop G M, Froguel P. 1995. A gene for maturity onset diabetes of the young (MODY) maps to chromosome 12q. Nature Genet 9:418-23.
Vionnet N, Stoffel M, Takeda J, Yasuda K, Bell G I, Zouali H, Lesage S, Velho G, Iris F, Passa P, et al. 1992. Nonsense mutation in the glucokinase gene causes early-onset non-insulin-dependent diabetes mellitus. Nature 356:721-22
Wang X, Li N, Liu B, Sun H, Chen T, Li H, Qui J, Zhang L, Wan T, Cao X. 2004. A novel human phosphatidylethanolamine-binding protein resists tumor necrosis factor α-induced apoptosis by inhibiting mitogen-activated protein kinase pathway activation and phosphatidylethanolamine externalization. JBC 279:45855-45864.
World Health Organization and International Diabetes Federation. 2006. Diabetes Action Now Booklet. http://www.who.int/diabetes/actionnow/booklet/en/ (Accessed Apr. 12, 2006)
Yamagata K, Furuta H, Oda N, Kaisaki P J, Menzel S, Cox N J, Fajans S S, Signorini S, Stoffel M, Bell G I. 1996. Mutations in the hepatocyte factor-4α gene in maturity-onset diabetes of the young (MODY 1). Nature 384:458-460.
Yamagata K, Oda N, Kaisaki P J, Menzel S, Furuta H, Vaxillaire M, Southarm L, Cox R D, Lathrop G M, Boriraj W, Chen X, Cox N J, Oda Y, Yano H, Le Beau M M, Yamada S, Nishigori H, Takeda J, Fajans S S, Hattersley A T, Iwasaki N, Hansen T, Pedersen O, Polonsky K S, Bell G I. 1996. Mutations in the hepotocyte nuclear factor-1α gene in maturity-onset diabetes of the young (Mody 3). Nature 384:455-458
Zhao J H, Curtis D, Sham P C. (2000) Model-free analysis and permutation tests for allelic associations. Hum Hered. 50(2):133-9.

1. A diagnostic method of determining whether a subject is at risk of developing type 2 diabetes, which method comprises detecting the presence of an alteration in the PEBP4 gene locus in a biological sample of said subject.

2. The method of claim 1, wherein said alteration is one or several SNP(s).

3. The method of claim 2, wherein said SNP is selected from the group consisting of SNP224, SNP225; and SNP 244.

4. The method of claim 3, wherein said SNP is allele T of SNP 225.

5. The method of claim 1, wherein said alteration is an haplotype of SNPs which consists in allele T of SNP224, allele T of SNP225, and allele A of SNP244.

6. The method of claim 1, wherein the presence of an alteration in the PEBP4 gene locus is detected by sequencing, selective hybridization, and/or selective amplification.

7. The method of claim 2, wherein the presence of an alteration in the PEBP4 gene locus is detected by sequencing, selective hybridization, and/or selective amplification.

8. The method of claim 3, wherein the presence of an alteration in the PEBP4 gene locus is detected by sequencing, selective hybridization, and/or selective amplification.

9. The method of claim 4, wherein the presence of an alteration in the PEBP4 gene locus is detected by sequencing, selective hybridization, and/or selective amplification.

10. The method of claim 5, wherein the presence of an alteration in the PEBP4 gene locus is detected by sequencing, selective hybridization, and/or selective amplification.


SEQUENCE LISTING (90 sequences)

SEQ ID NO: 1 - DNA, 901 bp, Homo sapiens
  CDS (93)..(776)
cccaaagtacttgtgtccgggtggtggactggattagctgcggagccctggaagctgcctgtccttctccctgtgcttaaccagaggtgcccatgggttggacaatgaggctggtcacagcagcactgttactgggtctcatgatggtggtcactggagacgaggatgagaacagcccgtgtgcccatgaggccctcttggacgaggacaccctcttttgccagggccttgaagttttctacccagagttggggaacattggctgcaaggttgttcctgattgtaacaactacagacagaagatcacctcctggatggagccgatagtcaagttcccgggggccgtggacggcgcaacctatatcctggtgatggtggatccagatgcccctagcagagcagaacccagacagagattctggagacattggctggtaacagatatcaagggcgccgacctgaagaaagggaagattcagggccaggagttatcagcctaccaggctccctccccaccggcacacagtggcttccatcgctaccagttctttgtctatcttcaggaaggaaaagtcatctctctccttcccaaggaaaacaaaactcgaggctcttggaaaatggacagatttctgaaccgtttccacctgggcgaacctgaagcaagcacccagttcatgacccagaactaccaggactcaccaaccctccaggctcccagagaaagggccagcgagcccaagcacaaaaaccaggcggagatagctgcctgctagatagccggctttgccatccgggcatgtggccacactgcccaccaccgacgatgtgggtatggaaccccctctggatacagaaccccttcttttccaaataaaaaaaaaatcatccaggaaaaaaa

SEQ ID NO: 2 - protein, 227 aa, Homo sapiens
MGWTMRLVTAALLLGLMMVVTGDEDENSPCAHEALLDEDTLFCQGLEVFYPELGNIGCKVVPDCNNYRQKITSWMEPIVKFPGAVDGATYILVMVDPDAPSRAEPRQRFWRHWLVTDIKGADLKKGKIQGQELSAYQAPSPPAHSGFHRYQFFVYLQEGKVISLLPKENKTRGSWKMDRFLNRFHLGEPEASTQFMTQNYQDSPTLQAPRERASEPKHKNQAEIAAC

SEQ ID NO: 3 - DNA, 701 bp, Homo sapiens
  misc_feature (501)..(501): r (SNP 181) is a or g
ggttggtctcgaactcccgacctcatgtgatccgcctgcctcggcctcccaaagtgctgggattacaggcgtgagccaccgcacccagcctttctttcttttttgagactgagtctcgctccattgcccaggctggagtgcagtggtgtgatcttggctcactgcaacctccacctcgcgggttcaagcaattctcctgcctcagcctcctgagtagctgggattacaggtgcctgccaccacgcccagctaatttttgtatttttagtagagacggggtttcaccactttggtcaggctggtctcaaacccctgaccttgtgatccacccgctgtggcctcccaaagtgctgggattacaggcgtgagccaccgcgcccagccagtttgtgattctttatgatacattaatgctttcattttcctaggggcttggatgtgcatggggctggaattggaaaaaatttatctgggcagaattttttcttcttccaaaatcarggtggatggttctgggcacatggctggtgctttttaggtacttgttgatctgcccgttggggtaactcctgtttcaccttctatgggaaggaagggaatatcccgtgggagtttgttttttttccttgactattctcatccttgtgattgcattctggctgggcagttagaaaggagggggtcagatgggccacttcccc

SEQ ID NO: 4 - DNA, 786 bp, Homo sapiens
  misc_feature (201)..(201): y (SNP 184) is c or t
cccaccacctccccgggagcctggctctgccatgggttcaagttgcagtaggatacccaggccaagggggttgcaatcttgattcctttctttttgagaaatgcagagagaagttatgtagttcctcgagaagcccccatcaccacccagccatggagactgctcccttccgcgtacccgtgccgtctcatggctggttcygaagggctcaactgggaacagcctcccaggctgcaacatagcagtgtgacacggagctcaaggcagcgtctcagtggtttgctcatttctgcacatgcttgcatctgtgtgtgcccttcctctatctagacagcacttctgtcacttctctcctggtaaacttctatttggtcctttaaaacacagctcagatgtcacctcctctgggaagccttccaagactacctaggcacagttatcaggcattttgattgtgctcttatcgttgcattattttcagggacaaacatcggttgggcacttattacaccctaagctttgtgctaggcacctcactgcattatctcattgaatacttacaaacctatggggtggatattcttacccccattttatagacagggaacctgaggcttagagcagttaaatcacctgcccaaggtcacctagctgttgagtggtgagactgggaagtgaacacaggcagtttgactccggtgccctccctcctaaccactaaattatattgcctcccttacatgacttaaaattgccccagactgtaattaaacgtttatgggtctg

SEQ ID NO: 5 - DNA, 201 bp, Homo sapiens
  misc_feature (101)..(101): r (SNP 193) is a or g
agtctctgggtggcccagagctaggcttcactgtcttcttaagaaatggatgctcaccttgtcaaacacttttgtgacagacatgattctccttcctccaraaaaaattcctctccgcctcccactctgctaaccctccaatgcaaagacagcacttcatttttaccgagttcaacatcttcggggacaggtgcaacgggc

SEQ ID NO: 6 - DNA, 401 bp, Homo sapiens
  misc_feature (201)..(201): r (SNP 194) is a or g
ccagaagaccagatgaatagagatctaagctgggtaatgttgctgcaccattgccctaaagggctgcctcttcacgcaggcaaccaacctccccaccccagacacacacagaaagaactggattccacgggcttctgggggtgcaaggggaaggggctgccatatctagtgggctcctggagaagctcgggctgaggagcrgagaagggagcagcctcagagcttctcatcttgtctccggtcctgcagtcttctttcctcacccgcaaatggtagagagaggagttttgagccgtacaaataaagcaaattcatatctgctcattgccatcctgtcctcatgctcaaccctctctcaaattttaatttggtagcttttagaaaggcaaatttatttgtga

SEQ ID NO: 7 - DNA, 601 bp, Homo sapiens
  misc_feature (301)..(301): y (SNP 204) is c or t
ctcaattctgaagtcatcccaccctaactgcttttgcactccggcctcaggaccgccgagcagagctcctaacaggaagccctgtgtgcctggggtggctgtgaatgtgaccttcagggcaatacttctctcttgagtgtttcacccctgagaccgctggggaggcctaagttcagaaaatgacaacgctctagccttggggggtgagggggagatgccagttctttctctttcatgcccactgctcctctccccttcctgatggctcaaacctcccagctctgtccctttgtgtgtttayggtgtttggccagggaagtgtgtggagtaacagaggtggctccagcactgcccacgaagctgctgcttgggtctcccttgaggaagccccttccagggccataggcagggctgcctctaaggatgcctgtccacctgagctccgtgggaggggccccggtcactgggccaccagctggttgccagggtaactaccctctcacccccctactcccacccagtggctgcgaaaaccctgccttgctcaggatagtcacaagctagttcagatttaaaccccttcctccacccacagttctag

SEQ ID NO: 8 - DNA, 1323 bp, Homo sapiens
  misc_feature (314)..(314): r (SNP 208) is a or g
ttcctgtattttgttcatgcactcagcatgcacatttattgtttgtctcatgtgtgtcaggcactgggccacagtactcagcagaggacactaagagccagccaccagtccctggcagagatcacaaggtggtgatgggagcaaacacaccaaaagccagtgcgatgtaactagcctttaacacagacattctcccaagcaactgattcacattttccccacattcttccaaccaagtctttgacccaagaggaacatgtgggtgttcctaaggtaactctgaaaatttctcaacaggcaggaattgctcacarttctcacccttccaattccctaacaaaagaaccaaggctgggcacggtggcttatgtctgtaatcccagcacactgggaggtcgaggcaggaagattgcttgggcccaaggagttgcagaccagcccaggcaagatggtaagaccccatctttaaaaaaaaaaaaaaaattaaaagaagaaaaaatttttttaagaaaagaaaagaaccatgttcttttgggacaccccagccaatagggtcacattttcatccacacctgtgtgtttaaaaaattctttgttaaaggcccaccagtccccactgaacctagtttcaaggaaaatctaggccacgatgctcagtttagagggtaatatgaagtaaggaagacaggcaaaatctatccgaaacctgaaccgtgttttacactgtgatcacggttagatataattcatctgattctctgagcctttctggaattttataaaatcttctgatgggtatggagaatagtatagtgtcccacacacagaaaaaaatttccccaacaaatgtcttatttacagcagaccggtcacattctgtggatttcttttaaatgaaaacacttccttgaaattggaagaacacttagaaatctcatggctgtcttcccttaattcatttctttctttccccaacaagttcactcctaaaaagctcaacattgcctccctcctcagccattccaattggggtttgggcgagggggtaaaaaacaccattttcagcctcacaggatgagatgggcccctcacgtggaagagctggcagtgggagggaaggaaccggaacaaatcacgagggtctgtcaggttgtggttctcagttatgccttgtttagcaccctatgggaagacagaaacccacacacactaggtttagaaaccagaggcttcaaccctgcaagtatttgttttgtgccaccagtagctattgtatttcacacaccaagtcttcatcatcccttgtagtccttttcgccagaa

SEQ ID NO: 9 - DNA, 601 bp, Homo sapiens
  misc_feature (301)..(301): y (SNP 211) is c or t
tcaaaaataagtgaataaacccagcagcacatggtagagatgttctagggaagcttcagagcctcctctgatcttgccattccatgagtctgtgtattttggcaggctggtcagctctacatccatctctgtggcctgctgggggaaacaccttgccatccttcaggttgagaaatccattctcaatatcacaggtattccacccacagtcattctgccctcatgccccataaggcgcctctcgcctccttaagccactatcaggcattattggtttagaaatctgtcctctgatcagatygcaggctcatctggacaagctgctcagctcctctctggacctggcacgggcctggcccggcacagaatgaatgcgcgaaatgactggcgtggtcagccaggccctggatgggcaaccttctgctcctgtattcctgcttgctggggttgggcccgcctaggggatagcttggatggcagagactgggacagccgctaagtgggggtacatctaaaagagctagagatgccaggagaaatgtccttcttctatgcggacctccatctttgggctgtttctggtgggttgcttctcctgtct

SEQ ID NO: 10 - DNA, 601 bp, Homo sapiens
  misc_feature (301)..(301): r (SNP 214) is a or g
gtggcattatgtgattcatgtccccagtgctgctgtcgcattctctgacatcctgggtgtcacctgccatgctgtcttgaatatgctacttggcctggggtgttactacaactcttcactcttcatatctactttttatgcaaataaaggttaaagatgtcaaaatctgatgcttctgattcctttcacatacagtcaactcaagacattcacatgtccatggctttaatctgtcatttgttgctgtggaatgcaactggtttgtgagtggggttgggagagaggacctcagtgtactccrtgaaggaggtggcacatgtgggcatttggtaaatgctgggtggaaagaaatagtgatcaaattagggtgggggcagtgggcacagaagagcttgtgtatgacttgtgtttcacagttcaccaaatatgtccacacataaggactcctggatcccccaggagccctgtgatagaggaagatatcatcctactctttaagaaaaaaaaaaaaaatccgtggcttagaagaaataaataatgggcttgaggacttggccagtgagtgattgagctgggatgattcttgttcttcttcttactg

SEQ ID NO: 11 - DNA, 801 bp, Homo sapiens
  misc_feature (401)..(401): r (SNP 217) is a or g
gttacaaagtggctttgagattggcagcagcctctaggtaagtgaggagctttgctggtgttcattttaactctaatttccataggagatgaaacctgactggtgacttcctttgtatttccctacatctgccaggacaaatctttttgcagagggctcaccaaaatatgtatattcactacatatctgcttctgggacactatcaggtctggcacccactcctcctacacaacatgcatacacaatagtattatagggggaatgataaaaataagtagtcttgccgtagggaccagtctcaggaacgtgcctggtggaaatgaatgtaatagtgccttgataagagccaggaatacaaaccacctccacgactgccagtaaaggacagagagataagcargggctggggcaatgagggagcaaaggagttaatgtcttttctccccgtcttcacttagggaatttgaattcttgatcaagcgatttgtcaggattgttgtgtatttactgtaattacagaacatcaaatgtgcattgacgtaattaccataaatatgtgtaaagacattataacgtgtaattatgtcatataattctttaataaagcatgcagaagaaaaattactctgcacatgctcaatgcatatcttctcctgactctttctactgaaggttttatttgctccctccccctccctcttttcaaattccagagacagtttctcccatttaggattaactagcagaagggtgagattagaatgtgtgtgtgtgtgtgtgtgtgtgtgtgtgcgtgcaag

SEQ ID NO: 12 - DNA, 601 bp, Homo sapiens
  misc_feature (301)..(301): m (SNP 219) is a or c
tactttcaataagtaaacctttgtcttaggctgagagaagcccagcctataaagcaaactacggcagcacctatctaccagggagcactatcacatggccatcagactccccccgtggagcctgcaccccaggagaaagtcaaagatggaaaatccaagtggcttctgaggcagagccagtaattcgccgagtgttgagcttgcagcactgaggttgcttacaactattcaattggaaaaaagtcagactcatcaggctggaatctcattagtttcccttagtgagctggagcttgaatamtccagggggatcagtccaaaacaaagccagcagaggaagcagccagaagtgtccaggtgcagtgggccgtctctggcccaggtgagatgagcctcgggacctatttagaagaagaggttgggttgggatagaactgagatagcagcttggcatactggcccacagagtacctgtgacggctgtctagtagccaagattgagaatattagaagacccacttcagcaatgtatgaaatcaaagcccacattttaaactccacactccctgacacggcatttgaggcccatctggtctccatc

SEQ ID NO: 13 - DNA, 854 bp, Homo sapiens
  misc_feature (362)..(362): r (SNP 222) is a or g
tcaagtagcttgaaaatggtggagtcaggatttggacatgggtctttttaactctatccattgtcagtagagttaagtcatgtgtgtactgcacagtggccctcaggcaagagggtgactaaaatccagaccctgtccccgttgccaagccacacctcctggcctagggatgaatcaggccaagagacaggacccctttttgcagtttgcacgaaggtgctgaggatgctggctgcagccctgtgcagtggaacctgctctctgggacttgccagggccatgtgtgccttttctgttaggacaggggaaagggaaagaaaatgaaagtcttttgttcagtacgtggaaaaatgaagaggcaraaaagtgatctttccaataccttaaatgatgaaggcactgccttcaatggttttaagtcttcctgactgtttactcttagctactttgattttctaggtaaaactggtttaaaatttttgtttttgagacagagtcttgctctgtcacccaggctggagtacagtggcgtgatctgggctcaccgcaacctctgcctcctgggttcaagtgattctcctgcctctgcctcctgagtagctgggactacaggcatgcgccaccacgcctagctaatttttgtatttttagtagagacagggtttccccatattggcaaggctggtctcgaactcctgatgtcaggtgatccgcccgcctcggcctcccaaactgctgggatcacaggtatgagccaccacgcccagcctaaattttgattcctgacttccagggtctttttactagatcagggaagattagacatagaaaggggaagattcttataggcaaaa

SEQ ID NO: 14 - DNA, 681 bp, Homo sapiens
  misc_feature (346)..(346): y (SNP 223) is c or t
atctcagctaggaagggaagcaaatatccccagtgacgatgaagaccagacatggaggtttaactgatctgtgtgaggccacagaacgagagtgtggcctcaacgctctcaatgttgacagaacagcactcaacatattgcattgcgacatgtctgcttctggactgcctatcccaccaggccgtgagcgccccagaggtgggaactatatcttagtcacccttgtactttcaagcctcacaatgggacctgccgaaaaaagggcactcagtaaacgtcttgtgaacgcatgaattggtgagtagcagagatgagatttcaagccagtggggagagaagaacaccyctcaaaaggaatgttgactccaagaaacatcaaaagagggaaagaaccttgagattttccaattccagctcttcaatctacagaagcaactgaggtccagataggatagatcacaggctcattcccatatagctaattggaggcagagccaggactaccaccgggctattgtcaccacgttatcccacgagggtcgagggtcggggaggacagggagcctgcccaggggtgcttcacaccgggggaggtgtgatttcaggccttaaggcacacacacgcttgaaaataaaactgtgtaattcaacaaacatctgcagcagcaatgcaattatctc

SEQ ID NO: 15 - DNA, 601 bp, Homo sapiens
  misc_feature (301)..(301): y (SNP 224) is c or t
ccatttacttcacagggcttttgtgagggtcaaatgacatgagaacttgctttgagaagctcaaagggctctataaatgtgaggtattgttatttcggcaggaggatgcagatgggggctatagatggccgccctggggttttaaaggaaaaacttgttcaaaaccgctcctgaggcagcgatcaccagctttacactcgcattgacttctctttcagggcagctgttggtgtacgtgttttgtttctgatgctgagacccttcctgaaccggatctccatcccttcactgagcaggtgcytactgtatggcaggtgcatgtgggctctggggacagctccaggtggcccgtgccctcctctccgccaggtgctctccctacttgcccttcccgataccttcctccccattctgcagccttctctcggccatgcagagatggatgtggagtggcccaggtggaccagtgagggtggggagggacccactggggctctggcaggggcagcctagacccagactctgctcaaagccactggttctccagcccaccaggagcttctatagacctcaagaccttgatgcagtccccaaagaaggg

SEQ ID NO: 16 - DNA, 909 bp, Homo sapiens
  misc_feature (409)..(409): y (SNP 225) is c or t
gaggtgggtgcttttctttcttgacctcacagatccaggatttggggtggtgtggagaatgtaccacgactttttgtcatgcacgcatggtgatgctaccatgccctattttgatcatgaacagagggaggcctcaaactggcagaaaagggaccgtggctcccggagagaaagcagccgcaaatgtgaaactatgaaggtgtattaatcgtgtctttttattctgtaatttttgtgctttgacattttggggcctcactgatcctggagggactgcccctcccagggctaattcctagaaataggaaactcctctgtgagaacaatttgcttatgaaaacctaccagtccagtgcccataccaccaaccacctcctctatcagatttttacactccaggccactatcyacctgcccaaaccaccccagggtgggtactagaccactaggaacagtccttatacccagagcctattgaaatgattcatactaaccaacccaaccctgctcaccctaccccacctgttcctcccacgatgaagactcctactcaggtttttccctagttccctctgcctcccaaccgagcctggggtatcccccatgtggccctgcgagacataccccctgcctctagggatctgtgattataaaacttcctccttcatgagagccatttctgtgtctgcatgtcttcccacacctgattaaaacaaatccccgacaccatcaaaacaacaggggcatctatagaaagcagggattatgatgcttgcttaaaggttcagagattcggacctggaaggatgcttatcacaattatccttcattatatccttgatccaaacactttctgatgtgagaacagccccaaggaggcagcagaaagtcccatcagtttctcacact

SEQ ID NO: 17 - DNA, 472 bp, Homo sapiens
  misc_feature (272)..(272): y (SNP 228) is c or t
gggtttgacttctgagagcctgagtttgcacagctacaaaatggggataattttcctaccatacaggatttgttgaaatcagagctcgggagagaagcacccattttggagctgggaaaaggaggaaaagaagaccatgggagtgagaaacagcaggaggcagaggctgggccatctgagccccctcaaaatccagaacaatgcaggggctgagaagaggaaggtggaagctgggctttggggagagaccccagtccccttcagaagatgcyaccctgttctcctttctgctctaaggctccggggagctcagagtttagggagatggtaaatgggagagaaaaattaaaggcaagagaagtctaaggaagtcgtcaaagcacagccaagcaaacagaacccttaaaggaggtaaaaggtaatgccgtaaattacagcttaaaagcctcgttttgggcaaattgctttttaa

SEQ ID NO: 18 - DNA, 701 bp, Homo sapiens
  misc_feature (501)..(501): r (SNP 230) is a or g
caggcgcagtggctcacgcctgtaatcccagcactttgggaagccgaggcgggtggatcacctgaggccaggagtttgagaccggcttggccaatatggtgaaaccctgtctcaaccaaaaatacaaaaattagccaggtgtggtggtgggtgcctgtgatcccagctacttgcaaggctgaggtgggagagttgcttggacctggaaggcagaggttgcagtgagccgagatcacaccgctgcactccagcctgagcgacagaggaagactccaccccaaaaaaaaaaaaaaaaaaaaaaaaaaaaaaaaaaaaaaaacccacccagtctatggtattttgttatggaaccccaagctaagacaccatcacagaaaatggtcactcaatcctcccagccagccatcagtggctcaatctaatactcaggttctcaatcaaagtgctccaagctctggtttgcggtaagtttagaaagataaccatagacattcatgcccragggaatgttcccctcacgccctgcacacccaagtgaaagctagaggtggccccttggctgttagctcctctttgtccctttctttatcctctggagttagtaagtccttctcgatgacctggcatagaggagatactccttgtaacagaaaatgtgtggttgaaatccaggctgaggggcagggcaggttgcgggatga

SEQ ID NO: 19 - DNA, 601 bp, Homo sapiens
  misc_feature (301)..(301): y (SNP 232) is c or t
aggacacattgctttttaaaattccaacttgtagctgcctcgatctataaatcaacccgttctctacaccctagagctccgagaagcattcgcaaatggcagcaagatatccatcctcccagaataggcataggaaagtgcccttgcaactgggccagggttacctggtacccagcctctccgcttccagggcatcctctttgacctccttcaaggcagatattgctctcccatggcaaagagcaggcaggcaagacagagccaatggtcaaggctccagctgggagtgggacttttgctyagatgcccaacttcagtgacctgcccctggactcagttgagttcctgtctttgttcatcatcacaaaaagaaaactaaagctgcttgccctgcctcctgccactgtgactgagaccctatttttagatgccaggctgagagtggggcaaggctgattaatgagaaacatcaaatactccacggaggctgggggaaccttgcttagagaagcagcaggctgcttcccggctccctggctgatctgcacacgaggcagcttgtgcctcagtacctgctgagggaccccagggctctccgtac

SEQ ID NO: 20 - DNA, 601 bp, Homo sapiens
  misc_feature (301)..(301): y (SNP 234) is c or t
tccaattccaaacctgaaattacccatcactctatttgcaagatgaagagtttgaggcagccgctgacattgcatatccccaaacaccaggttttcatgaaggagaaatgctatggaggtggaggaacctggggctctccagagatgcaggtcttcacttcagcctacccccacccctcactctccagggaccctagacacagaagtaccaggtgggagggagtgatgctgaacagagggtgttaccttaagacactgtccaggcagaggacactgcaggctgcctcctctgtccagcctygggagagtgtgccttcccacatccctatttcaatccagccttcaaacttctcactaaggctgcttggccagtttgccaagtcagttctgcaagggatttgtgcattcaccccacaggtttactgagcatctactatctgcatagccatgtgtccagccttgtaatttattcctcttcaaccagggataaagtatttccttcaaatctccctctcctctttctctctccttaaatctttaaaaaaacaaacaaacaaaaaacccctctgtcttgagtcccacatccactggccactgcctt

SEQ ID NO: 21 - DNA, 401 bp, Homo sapiens
  misc_feature (201)..(201): y (SNP 235) is c or t
tgggccttcttttcttgcaaatgaccatcccacccaggtaactaggggcttgaccctggaaggcacttccagttagcggccgagctttaaagcgttgcatcctggagatacttgcattttaatggcagaagggcatttccacaaccaagtggcaaagagagatagggcaggggttgcagagagaggtgaacccacccgacycaaatcagggatcctctatttttttcactcctgggtgacctgccctctgcaatgccttcctgcattggtggccccagccactcagccagctcacaggttcctggggtaggatgacggtcacctatctccccctggtgtcggctcttcccattgcagccccgggagatagagggcgatctccgcctacccgccagcgaggc

SEQ ID NO: 22 - DNA, 601 bp, Homo sapiens
  misc_feature (301)..(301): s (SNP 238) is c or g
tcaaaactcccctttaggccacaaccttctttcaccagtgagagcaggcaacaagtccctgtagcattcaaaatgctcttccttctaaatggctcccacaaaggccggcagctattttaaaatccaagaagcctttgctgacactcccaacataccgctgtgaacagatcccaaacatgtgaagagccctacactgatgcttgttcaatgcctttaaaacaaacaaacaaaaaaacaacccaggatgcaaatccattgagtgctcctttcaggtcccatgtgcccccttggtagatcagcscacacagtttttttcaaagaaacagtatttgatctgagaaacatctggaacctgtttccccagcacttggctgggagctttcagtggatgtcagcggcccaatcgctgagcgtgcaccctgcctgcctgagctgccggcaagatctatcggactgggggtggcggattgggtaagggggggcgctgtttgattaaaagctgctgggggcaggttcaggtctggcaggaagttctggaatgacagagaaggccacgaggataaggagccctgggagtgaagattaagactgggaatcctcc

SEQ ID NO: 23 - DNA, 601 bp, Homo sapiens
  misc_feature (301)..(301): r (SNP 241) is a or g
tttcctgtggcctcacccaacctctctggctggctgctgttgagcatagcagagcctacccgcaggtaaacctgtcctcctctcagagtccacgcctgctggaacactgaggatgcagacccaggtgcccagccctgccttctcttctggagtcagatggcagacctgcctctcgggcagctacctctggatgctacagcctcctcaaactctatgggtttctgtgcagaagtcggtgccaatggctgccatccctgtaaaaaacccagtgcttctctgtgtccccttcttggtgaaggcrccgttattagagttaacatataccccagcttgccagcgccatgcccaatgacacacattgtcctggcataactattaacagcatccccctttagtctcaaaagggtcctgtcttagatgagaaattatatactcaacctcattgttacccactcgcctgctcatcacagaaacccgagggtcatctggactctccctctccccaatatccccacagcccccaagctctatgggttccacgtccttaacaccttttaagcccagcccatggtccatgccatcacctaggttgcagggtggg

SEQ ID NO: 24 - DNA, 437 bp, Homo sapiens
  misc_feature (237)..(237): m (SNP 244) is a or c
atcagccagggatgtaggtggagcccagagggaggcagtcagggaaggcttcctagaggaggaagcattgtgacgggagcagggtccaaagacgccaaggatggggaagtggccaatgccagaatatggaggtgggaagctctatggtgggcacagaggcactgctgcacccctctttccagggctttggagtttgctgggggcgggggaggggggggtgttcccaggcatgtccamtggcagtcactgctttggtggcattgcttcagtcaccttggaagcaatgcaccatccccacatagcagtcaccaactcgtgcccttctccatcaggcccagaagaaataccttcatgctcttctctgtcctaaacactcaggctccccgcacccttttcttgagggacccaggtagcacttgctcctccagcctcaccag

SEQ ID NO: 25 - DNA, 701 bp, Homo sapiens
  misc_feature (201)..(201): s (SNP 245) is c or g
gtcactgctttggtggcattgcttcagtcaccttggaagcaatgcaccatccccacatagcagtcaccaactcgtgcccttctccatcaggcccagaagaaataccttcatgctcttctctgtcctaaacactcaggctccccgcacccttttcttgagggacccaggtagcacttgctcctccagcctcaccagtcatgscctctcccttaagggaagctccaactaccaccttccctaggaagaactatccttcctgcccacccccttccattctaaccagggaaagcttcccttcggccagcccacccctgcctgtgccagcgccccctggtgtctcccttagagcccttgccagtggcccactggccaggcccatgggctctggagttgggcagacctggctttgactctggctctggcacctttcagctctgtacttggccagattactcacttccataggacctgccttgcatttcctacctaacatcctttcacccttatctgatcaatcacaccttgacttgcattcagggcaaccatcctcctctactctcagtccttgtagattaaggggagctgtagagggtcactgctaggttcagggataggcacaagaccaaagcgagaccaatcctgtgacttttaatggatggaaaaccctatttccactggggttgacaaattggtcaaacaca

SEQ ID NO: 26 - DNA, 201 bp, Homo sapiens
  misc_feature (101)..(101): r (SNP 253) is a or g
aataaagacaaagctcctcttttgcgatgatggcttgaaatcaaaagacatcttcctggcatgcaaccttaaggtcataagtctctttctcagactggggrcctgctggtgtattctgggagtcctgagaatgggcccccttgactcgagtctgggagccctgaacctggggtgaaggttgtcagaagcagatagggaaag

SEQ ID NO: 27 - DNA, 601 bp, Homo sapiens
  misc_feature (301)..(301): m (SNP 255) is a or c
accgaggccagctccgaagggccagctccgaagggccagctgaggacagaagggtgcgtctgggtgcaccttgcagctgtgctgaactcagcactctcccaaaataatcgtatctggagaaggaggtctggggagctgcttggggctcagaaaggggtggcctgccccagcagtgcactgggaggaaggggagccggggactgtcgcctgcattcccttggggaacagtcctgccaccaggtgcatgtgggactctggtttgagcctgagtgggcagggagaagaataaaggcggcttgcmacaggctttccaatagctctgctctctgcttctcagaggcgggggtgggagagacgtgccagcaactcaggactcctggcttctgctctgagctctctctggatgctgggaggccactgtgtctccctagaatgcaccacttttccttttcacaaacaagtaaaataattcctgtcaccgtggtgccttgctaaggaatatgagatcatcgctgtaaagtacacgcagctagacctttagagaaactactttcagcatagtctgctttctggctcaattcctgcctccaggatcccaacc

SEQ ID NO: 28 - DNA, 601 bp, Homo sapiens
  misc_feature (301)..(301): k (SNP 159) is g or t
tagctgggccgaggggtcatctagggccctctgccttccagctaacgaggggcaggactctgagatgataagaaccctgttcccaggtatagcttgttcttttcatctggggatctcaaagcactttgcacacatgagtcaccaacatcgctacatgctagccaaggagacggggttattaactcatgaagggaaaccaaactgaagggggtgttaggggtgctgaaaggactggaatggtggtggggaggcatggggcctctgtaactttaggctgggaatgagactgtagcagggaggkgtctcccaggggtccttgggtggggaagtcaagaggcctgcagagaaagcccagggaagttagatggcaaaagtcctttccttgcagatacctaagaatttattcaccgctttcttttctttttttctttctttcttttttttttttttgagacagagtctcactttgtcgcccaggctggagtgcagtggtgcaatctcggctcactgcaacctctgcctcccgggttcaagcgattctcctgcttcagcctcccaagtagctgggattacaggcgcccaccattacgccgggctaatt

SEQ ID NO: 29 - DNA, 601 bp, Homo sapiens
  misc_feature (301)..(301): y (SNP 160) is c or t
agattcagacattctcaactggggctgggctctcagtccccattgccctctggggagcaggcagctctgctgctgttcagaggctctgctaacatctgggggctacctctgcaacaggagccggaagctcccccagccactacgccccatggccatcgcatcacctgctgcctcggctcccacgagccggcctccctctcaggcctctgccagctttcaagtccatggctctttctcactgcctccctgtggtctgtttgtccttccaggctcttggaaaatggacagatttctgaaccgyttccacctgggcgaacctgaagcaagcacccagttcatgacccagaactaccaggactcaccaaccctccaggctcccagaggaagggccagcgagcccaagcacaaaaccaggcagagatagctgcctgctagatagccggctttgccatccgggcatgtggccacactgctcaccaccgacgatgtgggtatggaaccccctctggatacagaaccccttcttttccaaattaaaaaaaaaaatcatccagggcttggtgctttgtatcctagcttgtattcctttgaaaagcaacag

SEQ ID NO: 30 - DNA, 601 bp, Homo sapiens
  misc_feature (301)..(301): r (SNP 161) is a or g
gcgatggccatggggcgtagtggctgggggagcttccggctcctgttgcagaggtagcccccagatgttagcagagcctctgaacagcagcagagctgcctgctccccagagggcaatggggactgagagcccagccccagttgagaatgtctgaatctccttccccagcacctctgtttagcaaaactgccttctcgctcccgggcagaaaggaagcccagtcaagcgggctgcccacttcctctactaatgccctctccctgcaggctcccctgaccccaggacaggaggcttcacccrctactttccttcacacctgctcacccctgctgctggggctgagattggtggaagtatctgtgctcttctttcaatcttctcccctaaaccccaaacacctcctcgtctctgtcccaggacagggggtgcaccagtgtgccttccccatggcagaggtggaggggcagcccctggaccctggcgggggagctgggcctgcagaaacccagacggtgcccaggtcggaggggttcctcctcagggggagaaaacacaagtgtgtacagggaggcgcatcttctggggcaagtgtgaacaggc

SEQ ID NO: 31 - DNA, 517 bp, Homo sapiens
  misc_feature (317)..(317): s (SNP 162) is c or g
gcagtaacaccccacctatgtgttggtactactgtaagagcttgacacataattcgcttaatcctcacaccaatcccaaaaagggaggactgttcccattcccagttcacagaagggatgactgaggcacaagctggggtactggcaaggaagcttcgtagtggcagggatggggaaaggcctttgcagattccctcaaatcctattcgactcagtgactgcatgctcagatggggcccaggggggctggaaaacaaggggttccaggagttcttttgcagaaagggtggccaaggtgctgagaaagcctctcccgstggaggtgtactgactgcagccgggttctcctaggaggctcagtgtcacctgagtgtcctttcagaggggctgtgcctgagcctctgcaccctaggagcccagctggctggaaggaaaggaattatggtattatgaccctgggaacgagtgtgtgcacacacatgtgtatgtgtgggtgcacgtgtgtgtgcgcacgcgc

SEQ ID NO: 32 - DNA, 601 bp, Homo sapiens
  misc_feature (301)..(301): y (SNP 163) is c or g
gtgtgtttgtgtctgtctgtgtgtgcgctggtggggggcagtctgagggcgggggctggtctccaggcagggccccctttcctctcaggcctcttccctccatgcttcctgacaccatgtgctgctgtgttctgctactaggctggtggatttcctcccagcctcatccctctgcagcaacatctcctactcttgaagctgcgtggggctctgctgcccttggccccatgattccccttctttctcacccaccgaggcctgcatgccttcagatgccacaagcaagacttcaaccccctaygtggcaagtccctgcccgtctctcctctctgacccctgtgggtgggtggttccacctctgagctgccgaatttgggtgcctgtggtgctagggagcggcttatggctgcctggcccaggactcccattttgcagtgagcaagtttcccctctgagggccctgaggctgccctggaggtggtgacccaagcctctcttgcccgttagtttccagcacgttgacaccatcctgggcgctcggccttggagtggcttctggagcaagctgtggcatgtcctctcgtggggcagagaggggatt

SEQ ID NO: 33 - DNA, 201 bp, Homo sapiens
  misc_feature (101)..(101): r (SNP 164) is a or g
tgggaaggggagaatgagacgcccagatgggcagtggtgggcaggcagatgtgagcaaagaagtagttcagggacgtctggcagtgtgccgcacacaaggrcaatggtacaaggtgcaaatgaaatctgcctggaaggaaagatgtccccctcagaggggtgttcacttagaatcagagggagacagcccagcatgtcaca

SEQ ID NO: 34 - DNA, 601 bp, Homo sapiens
  misc_feature (301)..(301): y (SNP 166) is c or t
ttgcccagatcagactcaattacagagcacaatgacttgccaaagttcaggggcagctgcctgcacatgccatgcaaagtttcggggtactcttctccctggctgcagtcacctgctttgagaccctgccttacataccaacccacccaactggagcctctacactggtctgtgggacttacttctcaagggagaccacacctgatgccaacatctttagggcactccttccagttcactcccaagaaacaccagccttcgaccccagtctccaaggctcatgagtgggggtcaaggcccyaatttccccgtaaatgcctgaagcgtttgttccaccccagaattcaccccggtggtggctggaaggatggggaggtcttacctcgagttttgttttccttgggaaggagagagatgacttttccttcctgaagatagacaaagaactggtagcgatggaagccactgtgtgccggtggggagggagcctggtaggctataggtagaagcaggagagaggtgagcatcaacatcccatactaccgagggcagagctctctgccttcccatggtcctgctgacctccctggggccccagatc

SEQ ID NO: 35 - DNA, 690 bp, Homo sapiens
  misc_feature (277)..(277): r (SNP 167) is a or g
cctcagcaatacgtgtgtctgtcctctttccctggatcccctgaggtacccattccaaaaccccactcactgccctgtctcccaaaagcagccatacatcctcctgctctcccattttcctcctctcccatcctacttcttctcccatcctccttcctctcccatcctcctcctgcagagagagaagcaatcccgcagagcccacgtggcctgaagtgtgcccaagacataacctacagccctgcttgtctcttctttcagggcgccgacctgaagraagggaagattcagggccaggagttatcaggtgagtaagaccctagggaccccttcccccagccagggcatcagtgacgatcacaggaatcaaaacctcatggtgctgctttgtcgtgtgctagcaccagcctgagagttttcttggatgccctgatttcatctccacaactgccttttatggtcactgccatcagtgtccccattttaagaggataacactgaggcacagaccatcctgcgccatgtgaatggctagtaaagaatgcagccagcttcacagtgcagggggccatctgactggaggctgcttgtaacccctgtccacccccatccttcccccagctcccctctctcagatccctgccccaggaggatgcccccaacctaccttgaaagggatgctcaggtact

SEQ ID NO: 36 - DNA, 690 bp, Homo sapiens
  misc_feature (201)..(201): y (SNP 168) is c or t
taaacctttcctcattggtcagtcctggaggggctacaatttctaccatagaatcaacacctggtaggggctttaggaaactaaattctaaaaggtgagagagttgggctccagagagggaaaatccagaatgttcaaggaggatgaggccttagaccccaggcccttcgagatcagctaactcaaggtctcatgcctttygacctcagtgccatcctgtgctctttctgtgacatcatgacaatcccatcagttctatgggaggcatcattttccaaagaccatgattaaaatacggagatattatctggagggctggagctgcggtgaaaatgggggccctcccacccgattttagctcttctaaaatatcttcatgatggccaagtgcaacagctcacacctgtaatcccagcactttgggaggctgaggcgggtggattgcttgagccaaggagtttgagaccgaacagtttaggcaacatagcaaaacctcgtctctacaaaaaatgcaaaaattatctgggcatgatggcccatgcctgtagtctcagctattcaggggactgagctgggaggatcacctgagcccagggaggtgatcctgagccatgatggtgccaccgcacttcagcctggatgacagagtgagacactgtctcaaaacaaatatatggattagaatgtcac

SEQ ID NO: 37 - DNA, 401 bp, Homo sapiens
  misc_feature (201)..(201): m (SNP 169) is a or c
tgtaacaggctttcagctttattctccaggcaaccatccaactggttgatctcacaacatcccattcagaggaagggcagagatctttagccccattggacaggagagaaaccacagaagcttcagctcctcgagggtggggactacaccctacccatctttgtgacctatgagacgctcggcacacagtgggtatagtcmaagtccgtgggcgccctggcccagtgcccagcccttgcaacttgagccatccctacttaacaacctctatcctttgggtgtccccgtagggacagagagctgcattttctgaggtctttcatagacttgagggtccagcgacactcctccctcacctggggaaccccttaggtaaaggctctttggctggatttggtagg

SEQ ID NO: 38 - DNA, 601 bp, Homo sapiens
  misc_feature (301)..(301): w (SNP 170) is a or t
agatcacaaagaaataataattacaggcttaaataattgaggcctagaggccatgctggggggagcagagatgaaggaagagataactgcttgggggtagtgatcagagaaagttattgaaaggaggctagatatggggccttgaaggatatataggagtttttcagttctccagtagacaagataaaggagatttctcttaggttgagaaagctgcctaagcaatgtataaatgtgtgcatgagacatgggaagatttcaaatgacttgggtgtccgttgaagccagaaccagcatgtgwgtcttcaagaatctcaagagaaggctagaaatgcaggatggggataagactggaatggtaggcagtgcctctgatttctgagactcaaagctctcagcaacctaaaccctgggtccacagatcccaggcaaggcttcttctcttctccaaatgatcagtaactgtgtcccaggagggccaccagagttggttggctgagctgccttccctgtcaagagaggaggagggtgggccccagctgggggacactgcccacgtggcaggtcagagcaggatgctgctgaattttgtcatcgatgg

SEQ ID NO: 39 - DNA, 601 bp, Homo sapiens
  misc_feature (301)..(301): r (SNP 171) is a or g
atcctgcgaagtctcctggcagggccagcctcctgaggctgatggctgcgccgtgggcttggaggttactcagctgtgaatgttgggcctgccccgctgggcctatactcagccagggtgcgcagagcgagtggcacatcatgtttattaataacccttgatgaggggcattaatagctcctaatgacaccatctgagcctggcctccttccttctctcgctgctcactccttcctctttccctccatcccttgaagacacatgatcccacagacagacacaggcgggctcggctcaggaragcgagtcagaggtgttgtccaggaggtgggggctctggagcgggggtctggccttgggggtcagacagaggatcctgggttggagagcaagtaggatgcctctggctctgccatcaagtggaaacttggccagaaactgctcggtgatgggagcaagggaagcgcagcagtgggaagaattttccagaactggtgggaatgggagtggcagggtgtgagatgggagaagctggagctgggaccagaatgaggccaagggggcctaggggcaacctcaggctgggtccgagagggccagg

SEQ ID NO: 40 - DNA, 636 bp, Homo sapiens
  misc_feature (436)..(436): y (SNP 172) is c or t
caagggggcctaggggcaacctcaggctgggtccgagagggccagggatgggagagcacaagtcacacacacacacttgcacactcgtgatatactcacaaggcacatgctcacatccacactctcacatatacatacgttcacacactcacatacttgtagtatgctcacacacccacacatgcatgttcacacatgctcactcacacacatgctgtcacacatgcacagaggcacaagctcacatacatgctcatatatccacatgcacagacactcacatgcacacacatgctcacacacactcacatgtactcacgcactcctctcctagactgcctttcactgggtatgcctggggctgggcatggtggggaggaccagaagaagggggcatttgggaaggggttggtggccaggctgaagctgggaccaygcaattaggaaaagagggtgtgggaatctccaggcccctggcagggggccaggacaaacagacaactgattagcactgaggtctgtgagtgagacctaagctgctctgttacccacccagggggctgagagccaatggggctgggctgtatccgagcagaggtgggagaggaggcagtcgaccaagaggcagggtgggat

SEQ ID NO: 41 - DNA, 601 bp, Homo sapiens
  misc_feature (301)..(301): r (SNP 173) is a or g
gaagtgacctgaccagacctttgctctctaactccggctggacagaggcagcccagcagccccctagcatccatctggcttggggagtggtgccccagggcttatgagtgagctggaaccccatgtcctggaatcccgagtggctgtgactgctgactgatgcccccctccccacagttctggagagtggctcagcagtctccccgagctccggccctagcagtcctcccgtctcccctctgtctgggtaagaatggcccctcagcccctgaccctgacccatcctgttctctactgccarcgagaagatgggtactgtcttggcttactctgctctcctcatgtgccacctatgagatgaggtgtccttgggaacccaggccacaaaggctgggctggggggatggagggggagggggtgcccaacagccaagagcctcgactgtggctctttggggaaggctggtagcagcaggtgggagtcagattcctcgttctgttctctgctttgtctccctcctttgccctattttaggtttccatacttggctctggtgggaagagaaagcataatctaccctttggagcctcttctctccct

SEQ ID NO: 42 - DNA, 401 bp, Homo sapiens
  misc_feature (201)..(201): y (SNP 174) is c or t
tgcagaggaggaaccctgactgtcttggaagccagcgccaccgtccccccggcaggcaggagagcggggttgggggcagcccatactgcttggcttccagtccccatggggaacatcgcctgtttcagagtatgttgaaatgagtctttgcacaaccatgacaagggcccagctgttcctggtgacagtgcgcagggacaygaggagatgggaggaaaggggacagggtcacagcagagggagggacccaaagaggatcaagggacagctgctggctccccaggcctccctatctggggctcccctgcctccttccaatcaaagcccaccgtgctctaggtccagggcagggcctcaccctcgaagctaggccttcagaggccttcctctgaggagtctgg

SEQ ID NO: 43 - DNA, 637 bp, Homo sapiens
  misc_feature (588)..(588): r (SNP 175) is a or g
cacagcaggtcacactggcctgcagccacaacccccaggctgtgatctcaccaagctccgagaagccaaacagggtcaggcttggctggtccccgagtgagcagggagacctcacaagcaaaacacaggtgttactggtgattcagcgggccagccccaccctccacggagtctagttctgctcaggtgttggaattgcaactgggatgattcagggagctcctatcagtacctgggcagaggaaaggggcgtggtgctgccggcaggtgctgaatgctctttctggcctccatgtgtgacaagtgttgacactcttaagaatcagggtggatgcacttcctccctccctgtcccctgcccatttcctaaggctggttgttgtcagccattgacctaaattcctcactttgtctcaaccagataagggatggaggaggaaaaggcagctccggaggggcttcaggctcaggaaccatttagcccccactccaaacagctgtggtctggggctgccatcaggctcctggctcagcccccaggatggtcccttctggtttgggaacaagtaaggatgaaacgggtccatraaatgagggccgagggccgtcattagcctgttttatgagtacacagata

SEQ ID NO: 44 - DNA, 401 bp, Homo sapiens
  misc_feature (201)..(201): w (SNP 176) is a or t
tgacggccctcggccctcattttatggacccgtttcatccttacttgttcccaaaccagaagggaccatcctgggggctgagccaggagcctgatggcagccccagaccacagctgtttggagtgggggctaaatggttcctgagcctgaagcccctccggagctgccttttcctcctccatcccttatctggttgagacwaagtgaggaatttaggtcaatggctgacaacaaccagccttaggaaatgggcaggggacagggagggaggaagtgcatccaccctgattcttaagagtgtcaacacttgtcacacatggaggccagaaagagcattcagcacctgccggcagcaccacgcccctttcctctgcccaggtactgataggagctccctgaat

SEQ ID NO: 45 - DNA, 401 bp, Homo sapiens
  misc_feature (201)..(201): k (SNP 177) is g or t
aatcaccagtaacacctgtgttttgcttgtgaggtctccctgctcactcggggaccagccaagcctgaccctgtttggcttctcggagcttggtgagatcacagcctgggggttgtggctgcaggccagtgtgacctgctgtgcagggagtatttttaaagagatacttgccctgtgttcccaggatcagctccctgagakactcaagttgttctttgaattctgcctccctctcccaatttgattgtagacattggtcaggggaacataaaatcacttgggataaagtgtgtagtggaaattaacatgcaatcttcttgccatagacaagcccctgggggatgcccagggagtcttggtgactggtagcacctgagggaggtgggatgagcagaatcaga

SEQ ID NO: 46 - DNA, 601 bp, Homo sapiens
  misc_feature (301)..(301): r (SNP 178) is a or g
ttggtgagatcacagcctgggggttgtggctgcaggccagtgtgacctgctgtgcagggagtatttttaaagagatacttgccctgtgttcccaggatcagctccctgagatactcaagttgttctttgaattctgcctccctctcccaatttgattgtagacattggtcaggggaacataaaatcacttgggataaagtgtgtagtggaaattaacatgcaatcttcttgccatagacaagcccctgggggatgcccagggagtcttggtgactggtagcacctgagggaggtgggatgrgcagaatcagactccctaattcccttgaggatgcagagatagactcacctgttttctcctgagttctagaactgcctaactgtggtcttccggtcagagcctggacaggagccacaggccaccatgacagggtctgccggtggaaaaagactcatgtatgagtcagcctgctgcctcggttcccagccagatgcgggcccagccctgcctccaggccatctgccgaaaattggggtcagtggagggcagaggagcccacgcatgcaggttcctcgtgcccctaaccaagctccccttttc

SEQ ID NO: 47 - DNA, 601 bp, Homo sapiens
  misc_feature (301)..(301): m (SNP 179) is a or c
gggagtcttggtgactggtagcacctgagggaggtgggatgagcagaatcagactccctaattcccttgaggatgcagagatagactcacctgttttctcctgagttctagaactgcctaactgtggtcttccggtcagagcctggacaggagccacaggccaccatgacagggtctgccggtggaaaaagactcatgtatgagtcagcctgctgcctcggttcccagccagatgcgggcccagccctgcctccaggccatctgccgaaaattggggtcagtggagggcagaggagcccamgcatgcaggttcctcgtgcccctaaccaagctccccttttcatgtcctctcttcttgcagacatcgccttcttacaatgcagaacagacgatgcactgtctcaggtcacgcttagcctcctacgggggcacgacagaagaagaggaacatttagagaggcacagctcctacatccttggatggggagagtggggagcaaacaggccaggtgagtcacttgggggttgggccatggttgttagacagtgacagcagtcctggacccgtatccagcactctccactctaggggtttcaggcc

SEQ ID NO: 48 - DNA, 601 bp, Homo sapiens
  misc_feature (301)..(301): r (SNP 180) is a or g
actgaggacttgtgatcctaatttcccaaccttcagctgctcagggtcaagtccttaggctccacttgctcttctcccctccccagcattcttctgtactaggttgcacatgatgagggtgatctggggggagaacaaggagggggtgtgactcagagttcacccggctgtcagcagccctgagtcagagggagggtggggaggtgggtggaacagcttttggtagtcttggggttccctataagcaagagcatcccagaaaaatagaagagagccccccagaagctacgaggctcccacrtgcagggtaggccttttctcggccaccactacacaccccaggagaacaggagaggcctccacgagtcctggatgatttaggtgcggacttgcctgggggaagggtgttggcccagaggaccccttgggtacctctgaactctgggattctgcagagaggcctcaccacatcaatgctttttgctctgtgtacccagatgggtctgtggagaatttggggggttccactgctgggactggggagatgacaatctggggtcaggggactgaggtgggtgtgggatgtgggaacaggaggggt

SEQ ID NO: 49 - DNA, 701 bp, Homo sapiens
  misc_feature (501)..(501): r (SNP 182) is a or g
ttgcctggagtccaaagatctgcttgagtgattcttaaatgtttctcaagcttctgctcctggctttctattatctagcggagtgtaacagaaatgccaataggcaggaaggaagagctgaagttcccagggctttgggccaaaatcgtatactcatttctctccctctttttttttttttttttttttttttttgagacggagtcatgctctgtcacccaggctgccgcgcagtggcgtgatctcggctcattgcaacctctgctttccgggttcaagcgtttctcctgcctcagcctcctgagtagctgggattacaggtgcccgccaccacactcagctaatttttgtatttttagtagagatggggttctaccatgttggccagtctggtctcaagctcctgacctcaagtaatccatccaccttggcctcccaaagtggtgggattacaggcgtgagccaccgtgctggggcaaaattgtatactcttttctaacrtcatttttgtcatcatcatcaccatccattactgataatgttagacatttattgttcttttttttcagccatgtgcagtgggtaagtacatcagcaagcaaactggcctcaagaatcccgccctatcctgggcatctggggttgaggctggctgtgccaacctttgcacagggtttcaatctgacctcactcagaaaacg

SEQ ID NO: 50 - DNA, 1281 bp, Homo sapiens
  misc_feature (962)..(962): k (SNP 183) is g or t
tcctccctggccctggccccagcccccaccaacctccaccactgtcccatgctcctccccacgtggactggggatgcttggggaagcaagccttttctagatgcgtccagcccccaatctttgcatcccttcctgccttaaaaagatgccaggagcagattgtccatttctgtctgtacaaaaacccacttgcctgggggctcattgaggcccttcttttgctcctgggtaagggagaagaaagtggttgctggcaggctgggaatttcggcttgccaaggctgggactgttgagttcggaaatcgtggtggccaaactccaccatttaccaggggaaagggacccagggagtacctgacctccctcagtctctggacctaggacccctggccctgtgggtcgcaggttccctggatgatgctggggaaaaggcctgtgttcattgcctcagttctaatttttctttttcaaaaggaagctctcagaacgcccagctatccccactgcagaaggctctccctgaaagggcacaggtggaggacgtttgcatttatctggagacagttcatggagaaacacaaagaggtgtcctgacaagacaacaggtccacagcagtcgtcatcatgagtgctcaccatatgccagggttcatcttttaaaatccttaaccaaacaaccaaactgtggttgggattattctctttttataggtgagaaaactgaggctgaaagccattgggtggtttgccaaagctgagaagtagctgagctggggtttgtactcacaccacggtgactcggaggcccaagccagttctgacacactgccttcccatgacattcccacgacaggttcccgaggtcgttggaatgcttacacatttcagcaacttcatggcaactgcccagctcgacccagaagataatactaccttgtactccccctgccgcttccttcckccaggcagctcacagacatgacctcattccctctcccagagtcccataggggtgaaggacagggtggggatcgttaactctgcttcctagggaggaaagggaggcccagagagggatgctggcccaccctgggtcatccagccagctagcgggagagctgcaactggaaatcaggcttccctgccctcggggacagttatttagcctcctggatgtgagctagaatcacccctaagatgcagcaattggggtactcctcttggggtactgggactcagtggctgaaacagctccaagagagttgggcctgtgctgaaaa

SEQ ID NO: 51 - DNA, 201 bp, Homo sapiens
  misc_feature (101)..(101): y (SNP 185) is c or t
gactccggtgccctccctcctaaccactaaattatattgcctcccttacatgacttaaaattgccccagactgtaattaaacgtttatgggtctggtaaaygcctcacaagcagagaccatgacttatttatctttgctgccttagaacatagcacatagtacaatgcatatagtaggtgcttggtggatgcttatgggat

SEQ ID NO: 52 - DNA, 1001 bp, Homo sapiens
  misc_feature (501)..(501): y (SNP 186) is c or t
ggctggagcgcagtagcatgatcttggctcactgcaacctctgcctcccgtgttcaagcgattctccgacctcagcctaccgagtagctgggattacaggtgcatgccaccacacccagctaatttttgtatttttagtagagacagggttttgccatgttggccaggctggtctcgaactcctgacctccggtgatctgcccacctctgcctcccaaagtgctgggattacaggtgtgagccactgttgcccgccggaagaagtcttaaaccccttgggtttcttcttgaatcccactccacaatatccccaaccaaagacttgttcattcttggctctggacactgcctccaatccccgacagcggtcagatcatcactgtgtagctctcatggtttaaagtacgtgtttacgagcagtgaatgctggccctagagcgtgtagcttcgaatcttgcactgccacttcccggatctctgaccttggctaagagacttagyatttctttgcctctgtttcccccatatctacaaaatgggaacaatgcgagtgtctgcctcacagggtgttgtgaggatgaggctaggtaatatatatgtaagaggtgcccagcctactaggctaattaaggttagctatcattattgttagaaagtttgccttctcaaccgagctctttctcccccaaacaactaccatgaaagaactatctcacctccaagaactaccatggttgtagttctgtctggggccccatggaagaagattaatttttctcctacatgactgctttcaactatttcattcattcattgcacagttttgctaggtggtctctggatgccaggtgcataatgccaggtgtggagagagccatgaagaagacctggtccctgctttcaagagcccatactccagaaggacgtgtaatttgtcttcggagcttctcttccccaggggaaacgctcatcagtttcttcacatattcctcacttaacat

SEQ ID NO: 53 - DNA, 601 bp, Homo sapiens
  misc_feature (301)..(301): y (SNP 187) is c or t
ctctttctcctttatcacccttccccagtcacttttcaaattctgtcagctcttccttcaaaatgtcgccagcatatgtcccttctttttttaaaaaaataaattccattgtttttcttcgtatgaaagagaaatatgctcagtgtagaaaatttagaaaataccaataaccaaagagaaaaaagaaaagaaagccgtcatccttcctcccgccattgctgcagtcattattttgagacacacaagtcagtcccggcctgtgctcaggaacctgcatctcctctttgttttgtgactcccyattcctgccactctgctgccctcagggcccctgactctctcctcttgtccaccccgccgcctatgccacgctaaccttctcaaactctgccttcgaaatgcttcagtttcctacacgtcaaatcctaggcagccatcagcccctaggaagcaggcgcccctctgggtggaagcccacagggtaccaggtctatggcgggcacctttgtgtgaatgagaaagaaggctcctcttccttggggcagaggaagctctgggactgggtcagtggcttggtgggcagacagcaaggagcagtcaa

SEQ ID NO: 54 - DNA, 1001 bp, Homo sapiens
  misc_feature (501)..(501): y (SNP 188) is c or t
agggtgtgctagtcacactgaatagtgtaagctgacccccacaggactgaacacctaccaggcatttgataaatacctgcaggttagttaacacaatgcattgacagtaccatatataccttcttcaacagagaatttgttggctgggcacagtggctcacaccactgagcactttgggaggccgaggtgggtggatcacttgaagtcaagagttcgagaccagcctagccaacatggtgaaaccctgtttctactaaaaatacaaaaattagtccgacatggtggcgcatgcctgtagtcgcagctacttgggaggcagaggcaggagaatcactcgaacccaggaggtggaggttgcagcgagctgagatcgtgccattgcactccagcctgggtgacggagtgagagtccatctcaaaaaagaaaaaaaaagaaaatttgtatagattataacaaaaccatatgtgatgaaataataataatattgacaacactaagyaatcattggatggaactctgactccgtgcttggcaccattcaggtagaaggtgagacttgactccagaggcagggcgcagacactggaccaaattgaggactagctaaaacagggatggggtggaagctgctttccataagacatgcccaccagcatgctatgtcagtttaccattgccatggcaacacctgggcgttactgcccctttccatggcaatggcctggccacccaaaagttaccaccctttccctagaaacttctgtacgaactgccccttaatctatgtgtaattaaaataggtataaatatgactgcaaaattgccctgagctgctactctcagcacagtgcctatggggtagccctgctctgcaggagcagtcatgaagctataatgctgtcagagttttaacactgccgcttcaataaagctgttttcttctgctctaccactggttcacccttgaagtctttccagggtgaagccaagaaccctcac

SEQ ID NO: 55 - DNA, 601 bp, Homo sapiens
  misc_feature (301)..(301): s (SNP 189) is c or g
ccattacctctccagggccctggggaccactgggaccctccccttcttcatctctgcaccaaagtaatacttagaaagcctcgtgaggggcagcggtttctttgaagaacagaatcctgagatgctgacgccagcgctcacacccttcctagcagagagattatgccaaggtttttgggggtgcatgggtctccagaagccattgcaaacccttccctttcccctttcttcatttctctttttactcatagatgagatttggcagaaaacatgaaagttctgtagggcacagaggccaatstatgttgacgctgataaatttggtctttcaacagtaaaattatgtggtttgggggagctttccatggtgcctggcatgtgagtacctttgaacctcagtgttgatggttgcagggtctgtgggcctcctggttacgccatcggtcttgctctcagccttcaaaccactggtctttcgttccggctgccactctgtcttcttacatcctcctcccacgggtttctccatcagccgagtcctctctgcatttttgcttatcttttcttgatgctgtttctctccctatctgcaaacacttat

SEQ ID NO: 56 - DNA, 601 bp, Homo sapiens
  misc_feature (301)..(301): r (SNP 190) is a or g
gccacattgacaggacctcctggtgacagcctgaggtggccaggacacttaccccctggctgccagcctgctctagctccatcggatacagggttctcttctcactcgccctgcacggcactcctgttgccccagagccgggggtggtggctggggcaccgggaaatgttatgaatggcagaactgctccaagcccccagccttcctcttgaggaggaggagagaatccgaccatgaggtattttcctggcgctaaggataaccttgtaggaggcagagggaatccaattagacatgcaargctggggaggctgagggcgtgtgggagaaggctgcttttcactttctggcttccccgcgccctcaatgagaaattatagcttggctccttcgttacagcccccctccgctctcctctttcctttcccagggccgtcatcacactcagcttttcccaccaccagcccctgcacagagggggaaacagagtcccacaacgatgggacccgcgaggcagaagccctcctgggcagtgccatcacggttcccttcctgtctctagttgcccctggaatttgggggaggctgcctcctgtacccc

SEQ ID NO: 57 - DNA, 601 bp, Homo sapiens
  misc_feature (301)..(301): y (SNP 191) is c or t
ttttcctggcgctaaggataaccttgtaggaggcagagggaatccaattagacatgcaaggctggggaggctgagggcgtgtgggagaaggctgcttttcactttctggcttccccgcgccctcaatgagaaattatagcttggctccttcgttacagcccccctccgctctcctctttcctttcccagggccgtcatcacactcagcttttcccaccaccagcccctgcacagagggggaaacagagtcccacaacgatgggacccgcgaggcagaagccctcctgggcagtgccatcayggttcccttcctgtctctagttgcccctggaatttgggggaggctgcctcctgtacccctggagttggttgcccatctacccctgtctctactgcttgtcctgagcttatctgagctctgcagtgattgtccatctcgcccctctctgcttgggctcggcacaggtaaagcagcaaatgacctccttcctcccagacccaaagtccccaactgcccccggtggacagatccatgtgcttccatgtgagactggatcctgttgcccggaagatgccccacttagtcctctttagaggcagt

SEQ ID NO: 58 - DNA, 601 bp, Homo sapiens
  misc_feature (301)..(301): y (SNP 192) is c or t
tcatggtttagaagattatgtgttgacatgggaaaatgttaatgatatattgctaagtaagaaagcaggctacattgcagaatgtacagtatgatacattcattttcaaaatttttgattaggacacagaccaaatctttaattaatgtcattttttttcttttcttggcaagcaagaagagtattatctagaatattgtaactaaaaaatgaaacagaaagacagttatacactaagtgtgttcacaggaatttttaagtcattaggatggagagtaggctgtcagtgagtcaaggaagyggagaaattctgaaactatagaattttagaagcggaaatcacccctagagatcgtcgggcccaacctcgctttttttgagacgaagaaattcaagctcctagggaagtctagacagtgactagtcagagatgggggtgggtgccagcctgaaactagggtcgtccaaccttcagtttatgctggtacgtttgccacccctcagagagaccccgctgatacttaagaggtgactctattcctaagccccctgaggtggggctgtgggctcctcccttcctcccagcctctgaagacctatg

SEQ ID NO: 59 - DNA, 601 bp, Homo sapiens
  misc_feature (301)..(301): r (SNP 195) is a or g
cagcccagctcttcactttacctgggaggacaccgaggcccaaaggagatgaacagcttgactccaagtcagtgaactcattggcagaagagatcagggcaggtctaaaggtgatgtggtggcctttaagcagggttgggaaattaggaataacttcgttttgggagagaagaagggaaaggatattcggggaaggggatagtaagtgaagaggtttgaaggtaggagtaagtaagttgctgatggtgagcagagcagcagtgggtggtcagttggtggaggaggctgctggtagaccccrtagggtccatgtgatctgaaggaaccgggggaagagagtcttgctgctatccatcgcaggggcaatgatgcagtgtatcacgactgttcactgactttgagtcaaattgttcatctccctggtcctcagtgtcctgagatgagcgtatgaaaactcaggtggtcagagtgagtgccctgaccaggatgatggccagaaagggctggtgccaacatcaatagcaatgctctgggtacagggctggctgtgcacgggtgcaggagggaggcgaggagagtcttccccaggggctgggaaact

SEQ ID NO: 60 - DNA, 565 bp, Homo sapiens
  misc_feature (239)..(239): m (SNP 196) is a or c
ggatctgctgtacaacacggtgcctatacttagtagtaccatactgtacacttaaaaactgtaaagagggtggatctcgtgttaagtgacctcatcataataaaaaagaaagaaagaaagaaaaaatgtgcatctggggttgagaaccgccaatgtccggtctttatccaggcctgggcaaggagtctggcacacagactgtgggaggacttccaacacggggtaaaaactgccttgcmtttttgttgttttcaggagacagggtctcattctgttgcccagggtggagtgcagtggtgcagtcatagctcactgcagcctccaactcctgggcccaagtcatcctcctgcctcagcctcctaagtagctaggactacaggtgcataccaccaggcctagctatttacttttattttttatagatatagggccccactatgttgcccaggttgctcttgaaccactggcctcaagcaatcctcccacctcggcctcccaaaaagtgctgggactgcagatgtgagccaccgcacccagcccctgccttgcacttttaaaagatcc

SEQ ID NO: 61 - DNA, 1001 bp, Homo sapiens
  misc_feature (501)..(501): y (SNP 197) is c or t
tagctaggcctggtggtatgcacctgtagtcctagctacttaggaggctgaggcaggaggatgacttgggcccaggagttggaggctgcagtgagctatgactgcaccactgcactccaccctgggcaacagaatgagaccctgtctcctgaaaacaacaaaaatgcaaggcagtttttaccccgtgttggaagtcctcccacagtctgtgtgccagactccttgcccaggcctggataaagaccggacattggcggttctcaaccccagatgcacattttttctttctttctttcttttttattatgatgaggtcacttaacacgagatccaccctctttacagtttttaagtgtacagtatggtactactaagtataggcaccgtgttgtacagcagatctccaggacttaatcatcttacattactgaaactttacacccgttgaacagcaacgccccatttcctcctcccttcgcccagatgcatattcacatcccytgagaagccttaaaaataaggatgcctatgctccacccccagagattctgacttagctggtctgggtgcagcttccttggtgattctaacaaacggttagaacctccatccttcctggtctcccttgctgcctcatgacctcatctcttccatgctgccccgtgaccaccctgctgcagccacaccagtccccatctgattctggaacacgccagctccttcctgctgttccctcggcccggctgccctgtcctcgcagggttgcctggtgaattcccactctgctcacaggtttccttctctggggaactttctccccgctctccttctcatctccacggttctcaggactcaactccatcctaacactccccactctgtaatggagtcattgatttttctcctctgctcccctcaccagagagggagctccttaacagtaaggatgtgattgcatacaggtcagcacggaggacttttagggcaggtcagctactctg

SEQ ID NO: 62 - DNA, 785 bp, Homo sapiens
  misc_feature (501)..(501): m (SNP 198) is a or c
acatctctcccccttggccaaaaggttaccacaattgacttgttctaatgcctggctcagccaagctgtactgtgcttactgacagcatcttcctggtccaaggggtcagtgcatttcccaacaaagaaatctgagtggtcaggcatgatttgtttttggtgaatctatgctgactcctaatgtgcactgttttcttttctatattgtctcagaccatctgcttattaaactgtttttgaattccgatgttttgatgaggactgatattacacttaccagcctaaatttgggaatccacattttccttccctatttctggaaatgatttcgtgattgcaaatctaagctctttctggaccttggaggtgtaattcatttagcaatctcaactcatttaaagcaatggcatgtttgtggattgcagaagggttttttgtaagaggggacagagcaggtaacagtccgcagaggcctagaaggtttgagcagatctgagacamtagaagcagatcagcaggagaacacgagggttgtggggcaggcaggtttcagagcctcaaaggtcttgagtaacacactggggagaggggtctctgtctgagatactggggaatcactgcagttcttcaagtaggaaagcaatatgatcctatttagattttagaaaaatcatcccatgaaggaaagattcacatttttgtagacggtgagattataggaggggaaactcttagtagatgtttgcaataacaatctagagacatctataacaatcaagggcctc

SEQ ID NO: 63 - DNA, 721 bp, Homo sapiens
  misc_feature (501)..(501): y (SNP 199) is c or t
ttcatccatccatccaccaaccacccacctattcaacctccatctatccctctatccattcacccactcacctctattcacccattcacccatctcttcctccattctttattcattcattcatttcttcatgccttcattaattcaacatttaccgagggcacttaatgttctgggctaagtgctaagaatataagaagacacatttctgctctccaggaactcatggtctaagagggaaaagagactcataaataataatgagggcactaattgaggtttaactgagtgtggcagcagcacagcgggactgactaactcagtctgggggtggtagagcaagcctttcggcagaggtgacatctgagctgggccttgaagtaggaggagttgcctcacggagatgaggagggtgctcccaattcccacagcaatttgagtctaaaaagcccacttggagttgactaaaccattgagtattgctctctcattatctcctayatgttggccttgtctttccaactgggcagcagaaaaaccacattttaaatccctaacatgtactcaccagatttgatttgatttgatttttttatttattgtattttattagtagccacagtgctgagagcggtgtgggtcagacaatcaatcctaatggctacctgtcaagtctgatgcccactggtggcttctctccttcaaggtctttaaagcgata

SEQ ID NO: 64 - DNA, 924 bp, Homo sapiens
  misc_feature (501)..(501): y (SNP 200) is c or t
tccattacctctgtcttttatgaggactgcatgaggtgcttatgtaaaacacagcacttggcatcacttgccaagtgcatcaatgatgtttttcttattttccttcttcctctccctctctccttttcttcctcctttttattctctcttctttagttattgttgttgttaatatgttctatggatatgagctacaggaagagtatctgtggccaggagagacaatatgccaggtggagacactgtgggagatgccaaggggcacaccttacaggtcacctgcaccagccttctacccagtgggaaaatttcctgggtcacccatggataattactgtcattaataaaagccagctccttggtgggatatatgctcagttcttgtgcagttcaaattcttagaaagttcttcctcataagggaccaaaatgtgcttccctggaaaacttcccccactggtcctgattccgcctggtggagtccagcaagatctcttctgtyttccaacgagatttccttaactctttaaaggccattccttctcgtgtgtccgtagttgttccaataggctagcctttcctacttccttccgctgctgctgacctgatgtggtttctacagaacctgaccattcaggttgctttcctatgctggtcattatccctcttaaattactgcggccagaacagaagacattattccagtgtggcataaacaatgcaaacagatagaactttctaagaccgaggaaggatcaccatgcttcaaagatgctgcccacccttctccctctcccctcccccaccgccaaccccctcgcttcctaagagcatctgctcaggggtgggtgtctgaggctcaaaggacagtgaaaagcccgggttcctcatgtcagggcaaggatggatttgggctgttggtctt

SEQ ID NO: 65 - DNA, 841 bp, Homo sapiens
  misc_feature (501)..(501): k (SNP 201) is g or t
tgatgtttttcttattttccttcttcctctccctctctccttttcttcctcctttttattctctcttctttagttattgttgttgttaatatgttctatggatatgagctacaggaagagtatctgtggccaggagagacaatatgccaggtggagacactgtgggagatgccaaggggcacaccttacaggtcacctgcaccagccttctacccagtgggaaaatttcctgggtcacccatggataattactgtcattaataaaagccagctccttggtgggatatatgctcagttcttgtgcagttcaaattcttagaaagttcttcctcataagggaccaaaatgtgcttccctggaaaacttcccccactggtcctgattccgcctggtggagtccagcaagatctcttctgttttccaacgagatttccttaactctttaaaggccattccttctcgtgtgtccgtagttgttccaataggctagcctttcctacktccttccgctgctgctgacctgatgtggtttctacagaacctgaccattcaggttgctttcctatgctggtcattatccctcttaaattactgcggccagaacagaagacattattccagtgtggcataaacaatgcaaacagatagaactttctaagaccgaggaaggatcaccatgcttcaaagatgctgcccacccttctccctctcccctcccccaccgccaaccccctcgcttcctaagagcatctgctcaggggtgggtgtctgaggctcaaaggacagtgaaaagcccgggttcctcatgtcagggcaaggatggatttgggctgttggtctt

SEQ ID NO: 66 - DNA, 601 bp, Homo sapiens
  misc_feature (301)..(301): r (SNP 202) is a or g
tgtgggcagtgcaccataggcctggctgtcacttgcagcccagcatcacacctgggctgtggcctgggacggctccagggagtctgctctggttcaggaggtcacaggggatccctagaggtggaatgatgggactgaccaaaggcttccaggctccaacacgctttggtttcctcaaaagccagctggggtcacgttaggtcctccaggctgggcctggactcatctagtttcaaagggcaaaaggatgagctgtgttgggcactgtctagaattttcactttgcaagacttgagtccarttgtctaagggagtttgctaaatcataagtagctgctatggtcagatgtcaaagttgggggctgtaggggagataattacatttaccaaagtcctactattctgtgtcagaagacttaatctcattgaacgctgggaaatagatattatcatgcccattttaatgatgaagaaactgagcctcagaggcttctcctctttgcatatgtaatttcctttttccttgtctccactctgaagagtgaggatttctgtcagtttcacagaggcaaagttgcagtctcagagaggtcaaacaact

SEQ ID NO: 67 - DNA, 720 bp, Homo sapiens
  misc_feature (249)..(249): r (SNP 203) is a or g
cttttcaaagagggagccaagtgggacagcataccttatgtatctgtctctgcctgtcctagcctggccaggttcggagactggagcctagtcctgttcctgagttgatggaaacccacaggtataggaagttccaccccaagccagcctctgggggtctcgtaataccaccactgtgctgcagattccttccttggtctctagaatagaagccctgtgagggcagagccttcctcaattttgtttgcrtttgtgtcaccaatgctaacctagctgcctgatttacagcaataataatataagagcaagtaatgatgtagagtttactatgtgttctgggtattgttctgagcactatccttaaaataattcgcttaagcttcataacaacagtgtgaagtaggcacactactatctccagcttacgagtgagtatattgagacacagagaggttgagcaactagcccaagatcacacagctaataatggtagattaggaattcaaaccacagcagtctggctctggagtccttgctcttaaccactgtgctatactgaatagtgttctatatatatttcttgaattaaacgaacaaaggggtaccatgaattaatgcccggtccctactgccaggcatctaacttttggcttttatttaagttatccaaagttccagagctttatcctactccgtactaaaccctgccagcccctcttt

SEQ ID NO: 68 - DNA, 1001 bp, Homo sapiens
  misc_feature (501)..(501): k (SNP 205) is g or t
tatatgaggacttattttgtgtcaagaacggtacctatcacaaagaacaccaggattcattcatcccttcaacaagcctttgctgaacacctactgtgtgccagaagctgctctaggtgccaggaggcaacagagcacaagacagtaaggtccatactttcaggaagcttttgttctaggtaaaagacgcagataataaaaataaataaataagcaagggaacgtgtgagtagtgctatgatgaaaataaaacagggtgggatgagaggtgggaagtgttctggaaggaaaaaaatagctgctatgaaggtcttcaggtgggaaaaaagtaggctttgaggagcaagaaagagcaccattgtggccagggacaagcaagggggagggtggacatggcaggtgcaggggagggagcagaggccaggccacagggggctctctaggcctggatagggagtggggttttattcaaagtgcaacacagaactgtttcagggtttkgggcaggacagtgatatgctctgatttatgcttttagaggattcttttggctgctgagtagaaaatgaatgatctccaaaggccagagaggaggccaggaggccagttaggagactcgcagtagcatgggaaggatgttggtatcttgggctaggatggcagaaatggagacagaaacaaatagatgggttgggggagtatttttaggtaaaatcggcagggtttgctgatagattcaacggcacggcagtaaggagggggatggagaaagagaaattgaggatgaatcctgggtttctggcttgagcaactgggccattaaatggtacaaataagacttggagaggaggctgtttgaaggtggacattcagagttctggtttagatatcttgagtttgaaatgcccctcagaatataggtatgcatgtcggtctgtaattcagaggagagatcaggactgggtgaataaatttggtagtcattgatatagagatggcct

SEQ ID NO: 69 - DNA, 1001 bp, Homo sapiens
  misc_feature (501)..(501): r (SNP 206) is a or g
taacaaatcacaatactttgggaggatgatgatgacatagaaacagaattagatctggatttgtccagataaagttttaaaaaacagctatatggaggtatagtttacatataataaacttcacccattttaagtgtacatttcaatgatttttaaaaaaatgtacaaagttgcgcaacgatcaccataatctaaatttagaacatttttatcatcccagaaagaaattttgtaccatgtatgcagtcattccttattccccccgccagttccaagcaaccatgaatctactttctgtctctgtaatctcctttttgcccttcctggacatttaatataaatggagtcataatacttgtttttttttgtgttggcatcttttactcagcgtaatgtttttgaggttcatccatgctgtacatgtattagtacttcattcctttttattgttgaataatatttcattgtatggatagagaccacatttgtttatccaattgrtggacatttggtttatttccactttggctattatgaatgatgctgctgtgaacatttgctgtaagtctctgtgcaaacacttgttttcaattctcttgcttagacgtctagagtggaactgctgggtcatattgtaaatttatgtttaacattttaagaaactaccaaactgtttttcaaagtggttgctccattttacaattccaccagaaggttctagtttctccacatctttgtcaacacttataattgtctgtcttttgattgtggccatgctagtgggtgtaaactggtatctcatgtttttaatttgcatttccctaaggactagtgatgttgagttccttttcatgtccttattgactatttttatatctttggtgaaatgtctatgtcaatttcttgcccatttaaaaattggattatttatcttctaaaattggattatctatccagaaataattgtaataggtgtgtcccaaggagtaagggaaaatgga

SEQ ID NO: 70 - DNA, 851 bp, Homo sapiens
  misc_feature (351)..(351): r (SNP 209) is a or g
ctggggtgtcccaaaagaacatggttcttttcttttcttaaaaaaattttttcttcttttaattttttttttttttttaaagatggggtcttaccatcttgcctgggctggtctgcaactccttgggcccaagcaatcttcctgcctcgacctcccagtgtgctgggattacagacataagccaccgtgcccagccttggttcttttgttagggaattggaagggtgagaactgtgagcaattcctgcctgttgagaaattttcagagttaccttaggaacacccacatgttcctcttgggtcaaagacttggttggaagaatgtggggaaaatgtgaatcagttgcttgrgagaatgtctgtgttaaaggctagttacatcgcactggcttttggtgtgtttgctcccatcaccaccttgtgatctctgccagggactggtggctggctcttagtgtcctctgctgagtactgtggcccagtgcctgacacacatgagacaaacaataaatgtgcatgctgagtgcatgaacaaaatacaggaaatatataactaatggtaccaattgccaaggagtttaccaccaagtaagtgccttttccataaaaagcaaaccctggatgtataaattgatgtaatcagcaataaaagattctcgaagaaataatagctaccattagtcaagcccttttcaactacatgccaaacccctcatatcccctgacgcattttattctgcagccaacacaccagctcagcactatcatctccattttaaagaagagaaaactgaggcttagggagaagactgagtaacttgcccaagggcacatcataagtaagtagcaaa

SEQ ID NO: 71 - DNA, 634 bp, Homo sapiens
  misc_feature (196)..(196): m (SNP 210) is a or c
cactcctaggcaggcctacgatggttctctgcgaaggcagtgcaccaatatatgtgtgtttccttcccagtgaagcttgacctttcattgtcttttcaggtctcagcggccctttgggctggtgtctttagggaacagtctgtagccattcttatccttttccaagctgtcctcccagaactacatgcagggttcmttctagcacagggctgccaactttctctgttctcacatagcctcttggcatcagcctggatttatctgcaccaggtggcatttgcacatgcagagggcctccctgatcctctgattacctggaggcctcattgcacaattcctcttccatgaaggtgtagaataaataggcctagagttgaacccaaaagaaagcaacctagaagtgaatagagaaaggacccgtttgctgttttgttagttgttgtccctaagctgttccctatcaatgacaaaatatttggcaccaaaggttcttaattttgaaagtaaccttgtcaaaagcttttgaaaaactaaatacaatttcctttcccactaagttatttaccccctaaagaactctgacagatgaagtaggaatgattgtactttacagaaatcctgcttccaccccctc

SEQ ID NO: 72 - DNA, 601 bp, Homo sapiens
  misc_feature (301)..(301): m (SNP 212) is a or c
gccccataaggcgcctctcgcctccttaagccactatcaggcattattggtttagaaatctgtcctctgatcagattgcaggctcatctggacaagctgctcagctcctctctggacctggcacgggcctggcccggcacagaatgaatgcgcgaaatgactggcgtggtcagccaggccctggatgggcaaccttctgctcctgtattcctgcttgctggggttgggcccgcctaggggatagcttggatggcagagactgggacagccgctaagtgggggtacatctaaaagagctagmgatgccaggagaaatgtccttcttctatgcggacctccatctttgggctgtttctggtgggttgcttctcctgtctgtgtcctcagaatgccaccctgcatacactgtgtgcacagatcagggcatctgtctccctcccatggtcagtggcctctcacaggcatcaggggaggctgcacactgggaggcagattgggagaagcaacagcctggggagtcccgggccccagagttctggtcctgcacgaccagggctggctccttcctctccctgtgccaccttcttttgagtggaaaagg

SEQ ID NO: 73 - DNA, 601 bp, Homo sapiens
  misc_feature (301)..(301): y (SNP 215) is c or t
cagcaacacttggatccagtttccattgacagcttcacaaactgaagatcgagaggcgggggacctggaaaggcagcctctttgggacaacatccaaatggatcttgccctgggtgacagtctccactcctttcctctcctccacatttggcttgcatggtgcctctagtaagttctaacatgcaggactccacggagactcctgggcttctggaagacggatgcgaaagacccggagcccaaagccggaacatctggcttgagttccaggcgcaccagttattggggcactgaatggccyttctttgtgactttgcattccagttccttctgagtcgattcttgagaaggaatatactcccatcaaggaagtgctttggggccctcagtgaagcctacttaaggtacacacttgggattttggaactagatgatgagacccagagatcatctggtataaccttattataggacagagaaggaaactgagctcaggaaagctaagcaacttgcccaagaccacacaggaaaagcaaggtcttctaactcatgttgcggtgctctcccagggcgccacccaccacacattagagttactcgg

SEQ ID NO: 74 - DNA, 701 bp, Homo sapiens
  misc_feature (201)..(201): y (SNP 218) is c or t
atgcttgggaagatgttgacattactgtgaatgcagggaacttttgtaaagatctgaacaaaaagtaaaatcccccagatgagatctctaggggtgttcaagtggagcccagattgtcaccaagactagaacacatttaaagtccttccccagccctcagagagtctattagatgtggggtctgtgtcatgatctgtattytcacatcccttcctctggccctgcaggccttttggaggctgtgcacccctgaggttaagtcttctgcagagaaaattgctggaagtattatgtccctggaactcagcgttaaatcaaatgagaaagaatgtccttatgcgtggggaagaccttcccaatgatgatatcaaaatcaaaattataaaggaaaaagacttattagactatgcaaaaaggtacaatttctctacataaaaataccacaaatatagtaaaaagcagccaggcatgatagctcacgcctgtaatcccagcactttaggaggccaaagcggaagaattgcatgggcctagcaatttgaaaccagcctagacaacaaagtgagaccccatttttacaaaaaaattttaaaaatagccaggtatggtggtgcacgcctgtattgccagttacttaggaggctgaggcaggaggaggatggttgagcctaggaggctgaggctgaggctgcagtgaacca

SEQ ID NO: 75 - DNA, 1284 bp, Homo sapiens
  misc_feature (951)..(951): y (SNP 220) is c or t
atcctttccttgcatacaacctacactcttatgcctctctatcttgccttacatctttgtttttgtagatcccttcccaatctttttaaaaaatattgtctttgggaggctgaggcaggaggatcacttgagcccaggagatgtctgtccctgggcaacatagggagaccccatccctacaaaaaatttaaaaattagctgggtatgatggtgcacgcctaaagtctcagctactctggaggctgaggtgagaggacctcttgagcccagcagtcaaggctgcaaatgagctatgatcatgtcactgcactctagcctgggtgacagagtgagactccgtctcaaaaaaaaaaaaaaaagaaaaagaaaaaaaattatatatacacatatatatgtatatatatatacacatatatatgtatatatatatatacacatatatgtatatatatatatggtgccagaatacacatatcatgcaatttgccatcctaaccatttttcagtgtccaattcggtagtcttaaggacattcacgttgatatgcaactgatctccagaatgcttttcattttgcaaaactgaaactctttactcattaaacgactccctcttccctctccccccagctcctggcaacaaccattctgctttctctctatgaatttgactcctctaggtatcctgcctaagtggaatggtacagtacctgtctttttgtgactggcttatttcacttagcataatgtcctcaagttccatccatgtagtagcatgggccagaatttccttcctctttaaggctgaataatcttctactgtgtggatataccacgttgtgtttatgcattcccatcttttttgccctctcaaaatctgactctatcttcaaagcttaaaacccaggatacgtactccgtggagctttcctcaagaccctccgttacaagytagtcatgtacaaagatcttttgagcatctgttccatgctaggcaacagggacacaaggatgagtagccacagtctctcctgcacggtccatccaagcccagcttactgagcacactgcaggtgactcggtgacagccctgactgtccatgttcatgacacactgtgctgccacgaggtctacttaatggtttctgtattagtcatctcatttgtaagctcttgaggacccgggccacatctgatctcagtatccccagtgcccagcacagtatattcaacatagaaaatgctcaataaatatttgttaaaatagaattaaatttgcattcct

SEQ ID NO: 76 - DNA, 601 bp, Homo sapiens
  misc_feature (301)..(301): y (SNP 221) is c or t
aagggggcctgaccagggctctctgatgtcagggatctattttctgttttggctctcgttctgagtcttgcagcccatctggattctgaatttccttaggtccaaaggatgtcaacttgattaaagagaaatatcaagaactaagaacttctcttgacagagaggtatggggtaggggtggtggacaggagaaaatgaaaagaaacagaaaatgggttcagttgccacttataataacctagtctcaatcttgcgctttagtgataagatcttaggagcaatttgtgaggagagtttgagyggcatactggttggatcgggagcacgaagtcattctctctgtccccgctaagtctgaaccctgaattaatttctcctgcatgtttagctagcaccaagcgagtgatgccaacacaaaagcggccgcttgatcaggagtattgatggggagaagagaaagaaaagatcaaaagcactccggctgaactgaaggtatggggtgacaacagatcccagagaaagctggcagcattgttcaggaggccacaggagtcgggggcctgggatccatcctcaatcttcccctaaccacacaagtaac

SEQ ID NO: 77 - DNA, 401 bp, Homo sapiens
  misc_feature (201)..(201): m (SNP 226) is a or c
accttgaaggcttcttaaattccccaagtctagactttcttatctttaaaatgcagctgaaaacaatgcctgtctcatggaggtgtaagggttaaatggaatcatgtagcaagcattcagcacatgtcaagtccaggtcaaggccaacatgcaggggcagggagaggtgtaacctctttcagtttggagccatgcaggacmctgccatttgcagccctttggggtaagactgtagcccccaaacatcctgctgccccagagcagtggcccagcaggtgcactgagagctctggtgcacctggcatgccccgactcctcacacagcagcaacagggacagtgacagggacctgagatggggtggcccacctggctggcagctggcactgggcacattgccca

SEQ ID NO: 78 - DNA, 836 bp, Homo sapiens
  misc_feature (340)..(340): y (SNP 227) is c or t
cacacagcagcaacagggacagtgacagggacctgagatggggtggcccacctggctggcagctggcactgggcacattgcccagaagtgggggagttctcaggtaggggcagaggtgggtgtttgagggagcagttagcagcactgtctgctgtggcaggggtctcctgggtaaggtcagagggattcgaggaggcccagggatcaccccagggtggggtgggacgctgctcaagtcacaaagcaaagttgcagggtgtaggggaggggatgggactgttagtctaattgtgactcatctctccttatttttctccttatctcttgtatttattttttycaatcctcccaagttactgggccttgaggacactcctagtgtgtgtgtgtgtgtgtgtgtgtgtgtatgtgtgtgtgtgtgtgtatttctatatatataatttttcttttttttgagacagagtttcgctcttgttgctcaggtggaagtgcaatggtgcgatctcaactcactgcaacctgcatctcctgggttcaagcgattctcctgcctcagcctcccgagcagctaggattacaggtggctgccaccatgcccagttaatttttgtaattttagtagagatggcgtttcaccatgttggccaggctggtctcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgcttcagtaggtaactgagtgggagggaggttagcttttcactgtgacccttatctgtacacctgctaactcaaaat

SEQ ID NO: 79 - DNA, 701 bp, Homo sapiens
  misc_feature (501)..(501): r (SNP 229) is a or g
ttcccagagacatagaaaatatttttaaagacccaattcagatttgaagagataacaatttcattgtctgtgatgaagagtacactggatgagatttaacagaaggttaactatacagaataagatattagtgaacttgaagacacagcaatagaaactattcaaaataaaacacagagagaaaaaagactgaaaaaaacccaccagaatagcatatcagtgagctgtgggacatcttcaagtagccaaatatacatgcaataggagtccctaaaattcccttttagggaatattagaaaaaatattagaagacataatggctgaaaaatgtcttaatttgatgaaaactgtaaacccacagatcccagaagcccaaccaaagaaatgccaagcacaagacacatgagaaaactacaccaaacacatcacaatcaaattgcttaaaaccagtgaagatgagagcacaggttaccattagcaaatgtgtcactggcagcccrcggcatatagtagcaaaataatcttgattatcaagtgtgattacctagaacaaagtacccattgaagataagagtttggcggaattgcacgatagattattatgtcagaatgatgaatataaagatgttggaataagatgacttcttctgccagaagtgggaaaagtaaagaaaaatggttagaagcttgaggtttaaat

SEQ ID NO: 80 - DNA, 401 bp, Homo sapiens
  misc_feature (201)..(201): r (SNP 231) is a or g
aggaggagttaagtttccaacatgtcacagaggatcaaacagccctgagggttctgctacaaaaaagatcaagggagtttgtcaaaaggcaggtcccaggacccctactggagggagaatgcattggacatacctggaacttttccaggagaacagaagctccaaactcagggatcccagatcctgcagcatagccttgcrttcccacacgctgtaggggttggggagtggggtggaggtgagcgttgaaccttggacctttctcattacaccgtagggacctcataaataggagggccttgtaatagcctgacgggcctgaaaagatggcagtggtatttcaggtacaacctgaaacttggattccatctcgctctgatagccttctactcccatagctt

SEQ ID NO: 81 - DNA, 820 bp, Homo sapiens
  misc_feature (189)..(189): y (SNP 237) is c or t
ctgctgcagccccctcctctggcagttctcacctccccaccaggcagtgggggttttggactgcagtgggcacctcctcccagcagcagctccccacagcagagctgggagctccacccacctccttctctccacccctcacccacaagccggcaagttttaacaaataacacatgatactgctcttcyaaaaatctgccaaacctttctcaaaactcccctttaggccacaaccttctttcaccagtgagagcaggcaacaagtccctgtagcattcaaaatgctcttccttctaaatggctcccacaaaggccggcagctattttaaaatccaagaagcctttgctgacactcccaacataccgctgtgaacagatcccaaacatgtgaagagccctacactgatgcttgttcaatgcctttaaaacaaacaaacaaaaaaacaacccaggatgcaaatccattgagtgctcctttcaggtcccatgtgcccccttggtagatcagcgcacacagtttttttcaaagaaacagtatttgatctgagaaacatctggaacctgtttccccagcacttggctgggagctttcagtggatgtcagcggcccaatcgctgagcgtgcaccctgcctgcctgagctgccggcaagatctatcgactgggggtggcggattgggtaagggggggcgctgtttgataaaagctgctgggggcaggttcaggtctgacaggaagttctggaatgacagagaaggccacgaggataggagccctgggagtgaagattagactgggaatcctgcttatggagcaccct

SEQ ID NO: 82 - DNA, 201 bp, Homo sapiens
  misc_feature (101)..(101): s (SNP 239) is c or g
tatccatcgccccatgttgctaggaggatctgtcctgtgaagtcctgcataggaagtgtctttgcaacctgaacatctaactgcaaaggttagaaatgctsacaatcctcactcccctttcacccagtccagttgcacccagaggagccccggatgacctggctggggacctgggctagagtgggacacagctgagtgatg

SEQ ID NO: 83 - DNA, 601 bp, Homo sapiens
  misc_feature (301)..(301): y (SNP 240) is c or t
tggtgatctccccagggtggccccttaccctgtcctgactttaccttctgctcccagcccgcagtcctggaacgatccagatccagtggctcttccccagcctggccccatgcagcctctcccagcagtggagcctggtgactcctgctccccattcctgcctcttccctgcacctccctcctctgtgcacctttacttcttctgcaatgtccgtgacatcatcattccccacgctctgccccaaagtccttccaccctgcaatttcatgacattgcctcttccccctgcagtaggggctyggctggctgagctccagctcccagggccagcatagggcagccccctccagcccctgcggccacacctgcacccctcctgcccactatccccttcctcctcctcttccctcagaagcatctcctgcacctcccacccctgacttccagtcagtggccaggtcccattggctccaaaggagctggacaccctttgaaatatcctcctaactcccaccattcacaccgtttatcaccttggaatgagcatttccagtcgacccctcaacccctccttctgctgtattttgacactgccctttt

SEQ ID NO: 84 - DNA, 609 bp, Homo sapiens
  misc_feature (409)..(409): r (SNP 242) is a or g
acccgccccgacccccgacccaaccacaagcctcatccctcacctcaccatcctggctcccatctgtcccttgctgggtcccggctcactgacaccctgtgtccccatgcttgcccgctacctgtctatctccccagctatactgcaagctccgtgatagtgtctttcgtctctgaatcaatagcactgacatggtgcctgatgcaatgtgttcagtaatatttacgaagtgagtcaccgcccatccccaacaccaaatagtccatggcggctgcagtcccctgagccagtgggcctgcacttgaccgcctccacatgtaggagaggagactccacatgtagtctactggaagacgtctctgctccatccattagggtctgttcttctagaattgctgtgggtgttggragccccgtgtccttcctcggctaatcattttgtgactgcagcagcctgtatttttctgctctgtgtatacagagaagggttggtaggggcggtggacctgtgagttccgagctctcttatagagaggagagttccaggcctctagaaaatttgggttgaaacttgtgtctccaccatgggccaatgagggcgagaaggtg

SEQ ID NO: 85 - DNA, 874 bp, Homo sapiens
  misc_feature (347)..(347): y (SNP 246) is c or t
ctccagcctcaccagtcatgccctctcccttaagggaagctccaactaccaccttccctaggaagaactatccttcctgcccacccccttccattctaaccagggaaagcttcccttcggccagcccacccctgcctgtgccagcgccccctggtgtctcccttagagcccttgccagtggcccactggccaggcccatgggctctggagttgggcagacctggctttgactctggctctggcacctttcagctctgtacttggccagattactcacttccataggacctgccttgcatttcctacctaacatcctttcacccttatctgatcaatcacaccttgacttgcattcagggcaaccatcctcctcyactctcagtccttgtagattaaggggagctgtagagggtcactgctaggttcagggataggcacaagaccaaagcgagaccaatcctgtgacttttaatggatggaaaaccctatttccactggggttgacaaattggtcaaacacaaacctaaggctgttggggccatctttgtcacaagggaagactctatttacaaatgaaaacagcacagaaaaaaacaatcaagagacagattcctggtgacactgagcacctggatccagcctcgcctgaagttatccctggagttttcagttttgtgaaccaatgtttccccctcttttctggcttaagccgatttgagttgactttttgtcacatgccattaatagtcttgatgaaagaaatacttaacttggagacttaaatgaaaggtccgagtagtgcccaggaatggaataaggtggcactcactgaccactttggcaaatctctctgaatctgccttctcataggtc

SEQ ID NO: 86 - DNA, 401 bp, Homo sapiens
  misc_feature (201)..(201): r (SNP 247) is a or g
cttccataggacctgcccttgcatttcctacctaacatcctttcacccttatctgatcaatcacaccttgacttgcattcagggcaaccatcctcctccactctcagtccttgtagattaaggggagctgtagagggtcactgctaggttcagggataggcacaagaccaaagcgagaccaatcctgtgacttttaatggrtggaaaaccctatttccactggggttgacaaattggtcaaacacaaacctaaggctgttggggccatctttgtcacaagggaagactctgtttacaaatgaaaacagcacagaaaaaaacaatcaagagacagattcctggtgacactgagcacctggatccagcctcgcctgaagttatccctggagttttcagttttg

SEQ ID NO: 87 - DNA, 201 bp, Homo sapiens
  misc_feature (101)..(101): y (SNP 248) is c or t
acgccacatcctgaagtcttgtactgttaaacccatcctgcctcagcccgggggtggggtggggagaaaacggatgtggaacatagagatttatgaggaayacagagctgtggtcaagaaagcctgggagagtgaagattttacaaaggaagatttgatcgaagctgtgggaagaagaaaaacatagtattctactactat

SEQ ID NO: 88 - DNA, 401 bp, Homo sapiens
  misc_feature (201)..(201): m (SNP 250) is a or c
gactgtcagtatgaatgaaccagttcatgtatttgctccctgagaagaagggcccgaacagcaccagggttctaagaatgtggcttcctggaagggaagaaaatatgatgcagagcaaggcaacagagcctgcagggcagggcaggccgggctgggccatgacagcaccaggctggctgtaagtgcatgcttatatttgamactcaggaacacaggtaagatttttcaaagacattttttaaaactgtttttgcttgcccttctattataatttatctccatgacctaagtcctctgtagggttaacgagggttaagacatcactgagagagaaagggtgtgggtagagagtgggtgttgcaggtccccagcctttcctaacaccttgcagggctaagccg

SEQ ID NO: 89 - DNA, 601 bp, Homo sapiens
  misc_feature (301)..(301): m (SNP 251) is a or c
gtgccttgctgggggtgatatctaaggggaccctgtgaggatgtaatttggagaatagatctggaaagggagctccctgaacctggggcctgggagtaggccaagagcagcttcctggtggggaaccctgcattgcccctcccaccttccacctcccacccttccagtggctcctgaagtccagccttgggcaaaaggccgctgagcccccagcacctggcagccccggaccaggacacttcggtgggccagcgcccccagtgccagaagtcacatgctctggtctcagagagtggaagcmggatgagtcatgggcttgctgagcaagtttgtttcttctaactctttgcgcatttacagcaattcttggtggatggcaagattttcacagcttttggagaagttttaggtgaccagttttatcttcctagcatcatcttaaggaccgtttggtttgtttctgctgcaactccttgctgccgcagctagcgagatgcgaggcagagaggggacccaccaagggcagagagacagacagcaaacagcacaggatgggaaaactgagaggtagggaggaggtaaactgagggtggagggacgc

SEQ ID NO: 90 - DNA, 601 bp, Homo sapiens
  misc_feature (301)..(301): r (SNP 254) is a or g
gcctggggagtcctcaggatgacccacaagtccacacaaatgaaggcttgcttttctaaccggcagagttggtgaggagagagccgaacgccaggctttccagcttacccaaagtagaagcagcttgtgcccttggaagggaagaagccagcctcgtcacccaactccatccagtgccatgatccctaaagggccctgcacaaacgctcctgggtcctatcactgacaaaaacctctcacacaggaagggtctaatcatacaccttctttcattcaaatgcctctttgttggggttctggrgagagggggcattcaggctgcagggagggatggtctctctctgggtctctctctgagggtctcagcctcctgcccccacaaagaccccccaggtttggagggcccaagcagagccgaccacatacacagccctcctggtcctgggagaggtgcattctggaaggcaggaaggagtctgtttctaaatccctttctcagtgccatcctccatcaaggtggtctccagatctgacacgtgttctgccttcccataaaatcaggctcaggagatggtgctccttggttctggatatacccctc